(19) United States
(12) Patent Application Publication
(10) Pub. No.: US 2023/0138633 A1
(43) Pub. Date: May 4, 2023

(54) SYSTEMS AND METHODS FOR PREPARING BIOLOGICAL SAMPLES FOR GENETIC SEQUENCING

(71) Applicant: Universal Diagnostics, S.L., Seville (ES)

(72) Inventors: Kristi Kruusmaa, Ljubljana (SI); Arianna Bertossi, Ljubljana (SI); Primoz Knap, Ljubljana (SI); Marina Manrique López, Sevilla (ES)

(21) Appl. No.: 17/980,755

(22) Filed: Nov. 4, 2022

Related U.S. Application Data
(60) Provisional application No. 63/275,556, filed on Nov. 4, 2021.

(51) Int. Cl. C12Q 1/6806; C12Q 1/6886; C12N 15/10

(52) U.S. Cl. CPC C12Q 1/6806; C12Q 1/6886; C12N 15/1068; C12Q 2600/154; C12Q 2600/156

(57) ABSTRACT

The present disclosure provides systems, methods, and apparatus for preparing biological samples (e.g., plasma) for sequencing (e.g., DNA sequencing, e.g., third generation sequencing). Moreover, the present disclosure provides various systems, methods, and apparatus that employ this sample preparation technology in the identification of biomarkers for detection of a disease or condition. For example, in certain embodiments, the biological sample preparation method includes capturing fragments of cell free DNA (cfDNA) with capture probes, converting the captured DNA fragments into circular DNA, and amplifying the circular DNA by performing rolling circle amplification (RCA). In particular, it is presently found that by performing this sample preparation method, it is possible to more successfully distinguish true alterations (e.g., aberrant methylation status and/or genomic mutations) from technical/sequencing artifacts.

### CROSS-REFERENCE TO RELATED APPLICATIONS

This application claims the benefit of and priority to U.S. Provisional Application No. 63/275,556 filed on Nov. 4, 2021, the disclosure of which is hereby incorporated by reference in its entirety.

### TECHNICAL FIELD

This invention relates generally to methods, and systems for identifying biomarkers for detection of a disease or condition, such as cancer.

### BACKGROUND

Disease detection is an important component of prevention of disease progression, diagnosis, and treatment. For example, early detection of colorectal cancer (CRC) has been shown to drastically improve outcomes of those suffering from CRC through early treatment of CRC. However, despite the availability of current tools to screen for and diagnose CRC and other cancers, millions of individuals still die annually from diseases which are treatable through early intervention and detection. Current tools to screen for and diagnose diseases are insufficient.

DNA methylation is a control mechanism that impacts numerous cellular processes including, for example, cellular differentiation. Dysregulation of methylation, therefore, can lead to disease, including cancer. Accumulated changes in DNA methylation (e.g., hypermethylation or hypomethylation), especially when the changes are located in crucial genes, can result in cancerous cells. These changes in methylation status, if detected, can be used to predict susceptibility of a subject to developing cancer, as well as the development or presence of cancer and, potentially, other diseases.

The most common method for analyzing genome-wide methylation status of a given organism is whole genome bisulfite sequencing (WGBS). In this method, the methylation status of single cytosines of sample DNA is determined by first treating the DNA (e.g., in fragmented form) with sodium bisulfite before sequencing. DNA methylation is present in mammals mostly at CpG dinucleotides—a CpG dinucleotide is a region of DNA where a cytosine nucleotide is followed by a guanine nucleotide in the linear sequence of bases along its 5→3′ direction. In WGBS, sodium bisulfite is used to convert unmethylated cytosines into uracil, while methylated forms of cytosine (e.g., 5-methylcytosine and 5-hydroxymethylcytosine) remain unchanged. The bisulfite-treated DNA fragments are then sequenced, e.g., via a next generation sequencing technique. However, the sequencing method may have low resolution of short genomic regions and be prone to errors.

Thus, there is a need for improved methods, systems and apparatus for analyzing methylation status of DNA and identifying methylation biomarkers.

### SUMMARY

The present disclosure provides systems, methods, and apparatus for preparing biological samples for genetic sequencing (e.g., DNA sequencing, e.g., third generation sequencing). Moreover, the present disclosure provides various systems, methods, and apparatus that employ this sample preparation technology in the identification of biomarkers for detection of a disease or condition. Standard next generation sequencing (NGS) techniques may insufficiently cover target regions, particularly as GC content of regions may vary widely from region to region. For example, methylation markers may have high GC content while mutation markers may have low GC content. Under certain NGS sequencing conditions, variations in GC content may lead to over-representation of regions having high GC content and/or underrepresentation of low GC content regions. Steps taken to improve GC coverage of high GC content regions may, in turn, lower coverage of low GC content regions (or vice versa). In addition, current NGS sequencing techniques lack sufficient means for determining data quality of samples.

It is found that these sources of error due to current NGS sequencing techniques may be eliminated or diminished by using the sample preparation method described herein and sequencing longer reads of the cfDNA via third generation sequencing.

In one aspect, the invention is directed to a method comprising: capturing a subset of deoxyribonucleic acid (DNA) fragments of cell free DNA (cfDNA) with one or more capture probes; converting said captured DNA fragments into circular DNA; and amplifying the circular DNA.

In certain embodiments, the method comprises extracting cfDNA from a biological sample and converting the cfDNA prior to capturing the subset of DNA fragments with the one or more capture probes. In certain embodiments, converting the cfDNA comprises enzymatic treatment of the cfDNA (e.g., with a member of the Apolipoprotein B mRNA Editing Catalytic Polypeptide-like (APOBEC) family (e.g., APOBEC-1, APOBEC-2, APOBEC-3A, APOBEC-3B, APOBEC-3C, APOBEC-3D, APOBEC-3E, APOBEC-3F, APOBEC-3G. APOBEC-3H, APOBEC-4, and/or Activation-induced (cytidine) deaminase (AID))).

In certain embodiments, the method comprises adding control DNA molecules to a sample comprising the DNA fragments of cfDNA, (e.g., wherein the sequence, number of methylated bases, and number of unmethylated bases of the control DNA molecules had been determined prior to addition of the control DNA to the sample).

In certain embodiments, the biological sample comprises a member selected from the group consisting of plasma, blood, serum, urine, stool, and tissue.

In certain embodiments, the one or more capture probes comprises one or more methylation capture probes and/or one or more mutation capture probes.

In certain embodiments, at least one of the one or more capture probes targets a differentially methylated region (DMR) in a genome of interest.

In certain embodiments, the method comprises converting the captured DNA fragments into circular double stranded DNA (dsDNA) and/or circular single stranded DNA (ssDNA) by performing DNA circularization. In certain embodiments, the method comprises converting the captured DNA fragments into circular ssDNA and a portion of the circular ssDNA is complementary to the original cfDNA strand.

In certain embodiments, the method comprises amplifying the circular DNA by performing rolling circle amplification (RCA).

In certain embodiments, the method comprises sequencing the cfDNA using the amplified circular DNA to produce sequencing results. In certain embodiments, the sequencing step is performed using a third generation sequencing system.

In certain embodiments, the method comprises performing sequencing using nanopore sequencing or single molecule real time sequencing (SMRT).

In certain embodiments, sequencing the cfDNA comprises producing reads each having length of at least 900 bases (e.g., at least 1 kb, at least 2 kb, at least 10 kb, at least 20 kb, at least 50 kb, at least 100 kb, at least 200 kb, at least 500 kb, at least 900 kb, at least 1Mb or more).

In certain embodiments, the method comprises performing (i) methylation target evaluation, or (ii) mutation target evaluation, or (iii) simultaneous methylation target and mutation target evaluation from the sequencing results.

In certain embodiments, the method comprises determining that a subject has a disease or condition (e.g., or, determining that the subject has a risk of a disease or condition) based at least in part on the sequencing results (e.g., wherein the disease or condition is a cancer (e.g., colorectal cancer) or a pre-cancer (e.g., advanced adenoma)), wherein the captured DNA fragments are from a biological sample of the subject.

In certain embodiments, the method comprises determining that a subject has a disease or condition based at least in part on the methylation target and/or mutation target evaluation.

In certain embodiments, the one or more capture probes are selected and/or are used in a predetermined ratio to enrich for only methylated reads or for only unmethylated reads in one or more specific target regions, thereby reducing (or eliminating) non-informative reads and enhancing a disease-distinguishing signal against background noise.

In another aspect, the invention is directed to a method comprising: extracting DNA (e.g., cfDNA) from a biological sample of a human subject to obtain a DNA sample; adding control DNA molecules to the DNA sample (e.g., wherein the sequence, number of methylated bases, and number of unmethylated bases of the control DNA molecules had been determined prior to addition of the control DNA to the sample; converting unmethylated cytosines to uracils of the DNA in the DNA sample using enzymatic conversion; adding an index primer (e.g., the same index primer, different index primers) to the converted DNA (e.g., at least 50%, at least 60%, at least 70%, at least 80%, at least 90% or more); amplifying the indexed DNA (e.g., using PCR); capturing a subset of indexed DNA with one or more capture probes, wherein each of said capture probes are targeted to a pre-determined mutation locus or a pre-determined methylation locus; converting said captured DNA fragments into circular, single stranded DNA, wherein converting said captured DNA fragments into circular ssDNA comprises: binding a splint DNA segment to the indexed DNA (e.g., wherein the splint segment comprises a segment of barcode DNA, a first segment complementary to a first portion of a strand of the indexed DNA (e.g., the 5′ end of the strand), and a second portion complementary to a second portion of a strand of the indexed DNA (e.g., the 3′ end of the strand)); amplifying the circular, ssDNA using rolling circle amplification; creating a library of DNA from the amplified, circular ssDNA (e.g., using PCR); and sequencing the library using third generation sequencing (e.g., nanopore sequencing or single molecule real time sequencing (SMRT)) to produce sequencing results.

In certain embodiments, sequencing the library comprises producing reads each having length of at least 900 bases (e.g., at least 1 kb, at least 2 kb, at least 10 kb, at least 20 kb, at least 50 kb, at least 100 kb, at least 200 kb, at least 500 kb, at least 900 kb, at least 1Mb or more).

In certain embodiments, the method comprises determining (e.g., by a processor of a computing system) whether a subject has a disease or condition based on the sequencing results.

In certain embodiments, the method comprises determining the number of methylated cytosines of the control DNA molecules that were converted into uracils.

Features of embodiments described with respect to one aspect of the invention may be applied with respect to another aspect of the invention.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a flow diagram of a general workflow of hybrid capture based targeted methylation nanopore sequencing, according to an illustrative embodiment.

FIG. 2 is a series of library preparation steps, according to an illustrative embodiment.

FIG. 3 is an exemplary DNA segment obtained after hybrid capture, according to an illustrative embodiment.

FIG. 4 is a splint DNA segment used in methods described herein, according to an illustrative embodiment.

FIG. 5 shows integration of splint DNA with a fragment of DNA, according to an illustrative embodiment.

FIG. 6 is circularized single stranded DNA, according to an illustrative embodiment.

FIG. 7 is a block diagram of an exemplary cloud computing environment used in certain embodiments.

FIG. 8 is a block diagram of an example computing device and an example mobile computing device used in certain embodiments.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DEFINITIONS

In order for the present disclosure to be more readily understood, certain terms are first defined below. Additional definitions for the following terms and other terms are set forth throughout the specification.

A, an: The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

About: The term “about”, when used herein in reference to a value, refers to a value that is similar, in context, to the referenced value. In general, those skilled in the art, familiar with the context, will appreciate the relevant degree of variance encompassed by “about” in that context. For example, in some embodiments, e.g., as set forth herein, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or with a fraction of a percent, of the referred value.

Advanced Adenoma: As used herein, the term “advanced adenoma” typically refers to refer to cells that exhibit first indications of relatively abnormal, uncontrolled, and/or autonomous growth but are not yet classified as cancerous alterations. In the context of colon tissue, “advanced adenoma” refers to neoplastic growth that shows signs of high grade dysplasia, and/or size that is >=10mm, and/or villious histological type, and/or serrated histological type with any type of dysplasia.

Administration: As used herein, the term “administration” typically refers to the administration of a composition to a subject or system, for example to achieve delivery of an agent that is, is included in, or is otherwise delivered by, the composition.

Amplification: As used herein, the term “amplification” refers to the use of a template nucleic acid molecule in combination with various reagents to generate further nucleic acid molecules from the template nucleic acid molecule, which further nucleic acid molecules may be identical to or similar to (e.g., at least 70% identical, e.g., at least 75%, 80%, 85%, 90%, 91%, 92%, 93%, 94%, 95%, 96%, 97%, 98%, 99%, or 100% identical to) a segment of the template nucleic acid molecule and/or a sequence complementary thereto.

Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or include biological tissue or fluid. In some embodiments, e.g., as set forth herein, a biological sample can be or include cells, tissue, or bodily fluid. In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or broncheoalveolar lavages, aspirates, scrapings, bone marrow. In some embodiments, e.g., as set forth herein, a biological sample is or includes cells obtained from a single subject or from a plurality of subjects. A sample can be a “primary sample” obtained directly from a biological source, or can be a “processed sample.” A biological sample can also be referred to as a “sample.”

Biomarker: As used herein, the term “biomarker,” consistent with its use in the art, refers to a to an entity whose presence, level, or form, correlates with a particular biological event or state of interest, so that it is considered to be a “marker” of that event or state. Those of skill in the art will appreciate, for instance, in the context of a DNA biomarker, that a biomarker can be or include a locus (such as one or more methylation loci) and/or the status of a locus (e.g., the status of one or more methylation loci). To give but a few examples of biomarkers, in some embodiments, e.g., as set forth herein, a biomarker can be or include a marker for a particular disease, disorder or condition, or can be a marker for qualitative of quantitative probability that a particular disease, disorder or condition can develop, occur, or reoccur, e.g., in a subject. In some embodiments, e.g., as set forth herein, a biomarker can be or include a marker for a particular therapeutic outcome, or qualitative of quantitative probability thereof. Thus, in various embodiments, e.g., as set forth herein, a biomarker can be predictive, prognostic, and/or diagnostic, of the relevant biological event or state of interest. A biomarker can be an entity of any chemical class. For example, in some embodiments, e.g., as set forth herein, a biomarker can be or include a nucleic acid, a polypeptide, a lipid, a carbohydrate, a small molecule, an inorganic agent (e.g., a metal or ion), or a combination thereof. In some embodiments, e.g., as set forth herein, a biomarker is a cell surface marker. In some embodiments, e.g., as set forth herein, a biomarker is intracellular. In some embodiments, e.g., as set forth herein, a biomarker is found outside of cells (e.g., is secreted or is otherwise generated or present outside of cells, e.g., in a body fluid such as blood, urine, tears, saliva, cerebrospinal fluid, and the like). In some embodiments, e.g., as set forth herein, a biomarker is methylation status of a methylation locus. In some instances, e.g., as set forth herein, a biomarker may be referred to as a “marker.”

To give but one example of a biomarker, in some embodiments e.g., as set forth herein, the term refers to expression of a product encoded by a gene, expression of which is characteristic of a particular tumor, tumor subclass, stage of tumor, etc. Alternatively or additionally, in some embodiments, e.g., as set forth herein, presence or level of a particular marker can correlate with activity (or activity level) of a particular signaling pathway, for example, of a signaling pathway the activity of which is characteristic of a particular class of tumors.

Those of skill in the art will appreciate that a biomarker may be individually determinative of a particular biological event or state of interest, or may represent or contribute to a determination of the statistical probability of a particular biological event or state of interest. Those of skill in the art will appreciate that markers may differ in their specificity and/or sensitivity as related to a particular biological event or state of interest.

Blood component: As used herein, the term “blood component” refers to any component of whole blood, including red blood cells, white blood cells, plasma, platelets, endothelial cells, mesothelial cells, epithelial cells, and cell-free DNA. Blood components also include the components of plasma, including proteins, metabolites, lipids, nucleic acids, and carbohydrates, and any other cells that can be present in blood, e.g., due to pregnancy, organ transplant, infection, injury, or disease.

Cancer: As used herein, the terms “cancer,” “malignancy,” “neoplasm,” “tumor,” and “carcinoma,” are used interchangeably to refer to a disease, disorder, or condition in which cells exhibit or exhibited relatively abnormal, uncontrolled, and/or autonomous growth, so that they display or displayed an abnormally elevated proliferation rate and/or aberrant growth phenotype. In some embodiments, e.g., as set forth herein, a cancer can include one or more tumors. In some embodiments e.g., as set forth herein, a cancer can be or include cells that are precancerous (e.g., benign), malignant, pre-metastatic, metastatic, and/or non-metastatic. In some embodiments e.g., as set forth herein, a cancer can be or include a solid tumor. In some embodiments e.g., as set forth herein, a cancer can be or include a hematologic tumor. In general, examples of different types of cancers known in the art include, for example, colorectal cancer, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Comparable: As used herein, the term “comparable” refers to members within sets of two or more conditions, circumstances, agents, entities, populations, etc., that may not be identical to one another but that are sufficiently similar to permit comparison there between, such that one of skill in the art will appreciate that conclusions can reasonably be drawn based on differences or similarities observed. In some embodiments, e.g., as sort forth herein, comparable sets of conditions, circumstances, agents, entities, populations, etc. are typically characterized by a plurality of substantially identical features and zero, one, or a plurality of differing features. Those of ordinary skill in the art will understand, in context, what degree of identity is required to render members of a set comparable. For example, those of ordinary skill in the art will appreciate that members of sets of conditions, circumstances, agents, entities, populations, etc., are comparable to one another when characterized by a sufficient number and type of substantially identical features to warrant a reasonable conclusion that differences observed can be attributed in whole or part to non-identical features thereof.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., a nucleic acid residue in a polynucleotide) may be identified as “corresponding to” a residue in an appropriate reference polymer. Those of ordinary skill in the art readily appreciate how to identify “corresponding” nucleic acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a fragment of DNA in a sample from a subject may be described as “corresponding to” a gene in order to indicate, in some embodiments, that it shows a particular degree of sequence identity or homology, or shares a particular characteristic sequence element.

Detectable moiety: The term “detectable moiety” as used herein refers to any element, molecule, functional group, compound, fragment, or other moiety that is detectable. In some embodiments, e.g., as sort forth herein, a detectable moiety is provided or utilized alone. In some embodiments, e.g., as sort forth herein, a detectable moiety is provided and/or utilized in association with (e.g., joined to) another agent. Examples of detectable moieties include, but are not limited to, various ligands, radionuclides (e.g., ³H, ¹⁴C, ¹⁸F, ¹⁹F, 32P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents, bioluminescent agents, spectrally resolvable inorganic fluorescent semiconductors nanocrystals (i.e., quantum dots), metal nanoparticles, nanoclusters, paramagnetic metal ions, enzymes, colorimetric labels, biotin, dioxigenin, haptens, and proteins for which antisera or monoclonal antibodies are available.

Diagnosis: As used herein, the term “Diagnosis” refers to determining whether, and/or the qualitative of quantitative probability that, a subject has or will develop a disease, disorder, condition, or state. For example, in diagnosis of cancer, diagnosis can include a determination regarding the risk, type, stage, malignancy, or other classification of a cancer. In some instances, e.g., as sort forth herein, a diagnosis can be or include a determination relating to prognosis and/or likely response to one or more general or particular therapeutic agents or regimens.

Diagnostic information: As used herein, the term “diagnostic information” refers to information useful in providing a diagnosis. Diagnostic information can include, without limitation, biomarker status information.

Differentially methylated: As used herein, the term “differentially methylated” describes a methylation site for which the methylation status differs between a first condition and a second condition. A methylation site that is differentially methylated can be referred to as a differentially methylated site. In some instances, e.g., as sort forth herein, a DMR is defined by the amplicon produced by amplification using oligonucleotide primers, e.g., a pair of oligonucleotide primers selected for amplification of the DMR or for amplification of a DNA region of interest present in the amplicon. In some instances, e.g., as sort forth herein, a DMR is defined as a DNA region amplified by a pair of oligonucleotide primers, including the region having the sequence of, or a sequence complementary to, the oligonucleotide primers. In some instances, e.g., as sort forth herein, a DMR is defined as a DNA region amplified by a pair of oligonucleotide primers, excluding the region having the sequence of, or a sequence complementary to, the oligonucleotide primers.

Differentially methylated region: As used herein, the term “differentially methylated region” (DMR) refers to a DNA region that includes one or more differentially methylated sites. A DMR that includes a greater number or frequency of methylated sites under a selected condition of interest, such as a cancerous state, can be referred to as a hypermethylated DMR. A DMR that includes a smaller number or frequency of methylated sites under a selected condition of interest, such as a cancerous state, can be referred to as a hypomethylated DMR. A DMR that is a methylation biomarker for colorectal cancer can be referred to as a colorectal cancer DMR. A DMR that is a methylation biomarker for advanced adenoma can be referred to as an advanced adenoma DMR. In some instances, e.g., as set forth herein, a DMR can be a single nucleotide, which single nucleotide is a methylation site. In some instances, e.g., as set forth herein, a DMR has a length of at least 10, at least 15, at least 20, at least 30, at least 50, or at least 75 base pairs. In some instances, e.g., as set forth herein, a DMR has a length of equal to or less than 5000 bp, 4,000 bp, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp (e.g., where methylation status is determined using quantitative polymerase chain reaction (qPCR), e.g., methylation sensitive restriction enzyme quantitative polymerase chain reaction (MSRE-qPCR)) (e.g., where methylation status is determined using a next generation sequencing technique, e.g., targeted next generation sequencing). In some instances, e.g., as set forth herein, a DMR that is a methylation biomarker for advanced adenoma may also be useful in identification of colorectal cancer and vice versa.

DNA region: As used herein, “DNA region” refers to any contiguous portion of a larger DNA molecule. Those of skill in the art will be familiar with techniques for determining whether a first DNA region and a second DNA region correspond, based, e.g., on sequence similarity (e.g, sequence identity or homology) of the first and second DNA regions and/or context (e.g., the sequence identity or homology of nucleic acids upstream and/or downstream of the first and second DNA regions).

Downstream: As used herein, the term “downstream” means that a first DNA region is closer, relative to a second DNA region, to the C-terminus of a nucleic acid that includes the first DNA region and the second DNA region.

Gene: As used herein, the term “gene” refers to a single DNA region, e.g., in a chromosome, that includes a coding sequence that encodes a product (e.g., an RNA product and/or a polypeptide product), together with all, some, or none of the DNA sequences that contribute to regulation of the expression of coding sequence. In some embodiments, e.g., as set forth herein, a gene includes one or more non-coding sequences. In some particular embodiments, e.g., as set forth herein, a gene includes exonic and intronic sequences. In some embodiments, e.g., as set forth herein, a gene includes one or more regulatory elements that, for example, can control or impact one or more aspects of gene expression (e.g., cell-type-specific expression, inducible expression, etc.). In some embodiments, e.g., as set forth herein, a gene includes a promoter. In some embodiments, e.g., as set forth herein, a gene includes one or both of a (i) DNA nucleotides extending a predetermined number of nucleotides upstream of the coding sequence and (ii) DNA nucleotides extending a predetermined number of nucleotides downstream of the coding sequence. In various embodiments, e.g., as set forth herein, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb.

Homology: As used herein, the term “homology” refers to the overall relatedness between polymeric molecules, e.g., between nucleic acid molecules (e.g., DNA molecules and/or RNA molecules) and/or between polypeptide molecules. Those of skill in the art will appreciate that homology can be defined, e.g., by a percent identity or by a percent homology (sequence similarity). In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, e.g., as set forth herein, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Hybridize: As used herein, “hybridize” refers to the association of a first nucleic acid with a second nucleic acid to form a double-stranded structure, which association occurs through complementary pairing of nucleotides. Those of skill in the art will recognize that complementary sequences, among others, can hybridize. In various embodiments, e.g., as set forth herein, hybridization can occur, for example, between nucleotide sequences having at least 70% complementarity, e.g., at least 75%, 80%, 85%, 90%, 91%, 92%, 93%, 94%, 95%, 96%, 97%, 98%, 99%, or 100% complementarity. Those of skill in the art will further appreciate that whether hybridization of a first nucleic acid and a second nucleic acid does or does not occur can dependence upon various reaction conditions. Certain conditions under which hybridization can occur are known in the art.

Hypomethylation: As used herein, the term “hypomethylation” refers to the state of a methylation locus having at least one fewer methylated nucleotides in a state of interest as compared to a reference state (e.g., at least one fewer methylated nucleotides in colorectal cancer than in a healthy control).

Hypermethylation: As used herein, the term “hypermethylation” refers to the state of a methylation locus having at least one more methylated nucleotide in a state of interest as compared to a reference state (e.g., at least one more methylated nucleotide in colorectal cancer than in a healthy control).

First, second, etc.: It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner. In addition, unless stated otherwise, a set of elements may comprise one or more elements.

“Improved,” “increased,” or “reduced”: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.

Methylation: As used herein, the term “methylation” includes methylation at any of (i) C5 position of cytosine; (ii) N4 position of cytosine; and (iii) the N6 position of adenine. Methylation also includes (iv) other types of nucleotide methylation. A nucleotide that is methylated can be referred to as a “methylated nucleotide” or “methylated nucleotide base.” In certain embodiments, e.g., as set forth herein, methylation specifically refers to methylation of cytosine residues. In some instances, methylation specifically refers to methylation of cytosine residues present in CpG sites.

Methylation assay: As used herein, the term “methylation assay” refers to any technique that can be used to determine the methylation status of a methylation locus.

Methylation biomarker: As used herein, the term “methylation biomarker” refers to a biomarker that is or includes at least one methylation locus and/or the methylation status of at least one methylation locus, e.g., a hypermethylated locus. In particular, a methylation biomarker is a biomarker characterized by a change between a first state and a second state (e.g., between a cancerous state and a non-cancerous state) in methylation status of one or more nucleic acid loci.

Methylation locus: As used herein, the term “methylation locus” refers to a DNA region that includes at least one differentially methylated region. A methylation locus that includes a greater number or frequency of methylated sites under a selected condition of interest, such as a cancerous state, can be referred to as a hypermethylated locus. A methylation locus that includes a smaller number or frequency of methylated sites under a selected condition of interest, such as a cancerous state, can be referred to as a hypomethylated locus. In some instances, e.g., as set forth herein, a methylation locus has a length of at least 10, at least 15, at least 20, at least 30, at least 50, or at least 75 base pairs. In some instances, e.g., as set forth herein, a methylation locus has a length of less than 5000 bp, 4,000 bp, 3,000 bp, 2,000 bp, 1,000 bp, 950 bp, 900 bp, 850 bp, 800 bp, 750 bp, 700 bp, 650 bp, 600 bp, 550 bp, 500 bp, 450 bp, 400 bp, 350 bp, 300 bp, 250 bp, 200 bp, 150 bp, 100 bp, 50 bp, 40 bp, 30 bp, 20 bp, or 10 bp.

Methylation site: As used herein, a methylation site refers to a nucleotide or nucleotide position that is methylated in at least one condition. In its methylated state, a methylation site can be referred to as a methylated site.

Methylation status: As used herein, “methylation status,” “methylation state,” or “methylation profile” refer to the number, frequency, or pattern of methylation at methylation sites within a methylation locus. Accordingly, a change in methylation status between a first state and a second state can be or include an increase in the number, frequency, or pattern of methylated sites, or can be or include a decrease in the number, frequency, or pattern of methylated sites. In various instances, a change in methylation status in a change in methylation value.

Methylation value: As used herein, the term “methylation value” refers to a numerical representation of a methylation status, e.g., in the form of number that represents the frequency or ratio of methylation of a methylation locus. In some instances, e.g., as set forth herein, a methylation value can be generated by a method that includes quantifying the amount of intact nucleic acid present in a sample following restriction digestion of the sample with a methylation dependent restriction enzyme. In some instances, e.g., as set forth herein, a methylation value can be generated by a method that includes comparing amplification profiles after bisulfite reaction of a sample. In some instances, e.g., as set forth herein, a methylation value can be generated by comparing sequences of bisulfite-treated and untreated nucleic acids. In some instances, e.g., as set forth herein, a methylation value is, includes, or is based on a quantitative PCR result. In some instances, e.g., as set forth herein, a methylation value

Mutation: As used herein, the term “mutation” refers to a genetic variation in a biomolecule (e.g., a nucleic acid or a protein) as compared to a reference biomolecule. For example, a mutation in a nucleic acid may, in some embodiments, comprise a nucleobase substitution, a deletion of one or more nucleobases, an insertion of one or more nucleobases, an inversion of two or more nucleobases, or a truncation, as compared to a reference nucleic acid molecule. Similarly, a mutation in a protein may comprise an amino acid substitution, insertion, inversion, or truncation, as compared to a reference polypeptide. Additional mutations, e.g., fusions and indels, are known to those of skill in the art. In some embodiments, a mutation comprises a genetic variant that is associated with a loss of function of a gene product. A loss of function may be a complete abolishment of function, e.g., an abolishment of the enzymatic activity of an enzyme, or a partial loss of function, e.g., a diminished enzymatic activity of an enzyme. In some embodiments, a mutant comprises a genetic variant that is associated with a gain of function, e.g., with a negative or undesirable alteration in a characteristic or activity in a gene product. In some embodiments, a mutant is characterized by a reduction or loss in a desirable level or activity as compared to a reference; in some embodiments, a mutant is characterized by an increase or gain of an undesirable level or activity as compared to a reference. In some embodiments, the reference biomolecule is a wild-type biomolecule.

Nucleic acid: As used herein, in its broadest sense, the term “nucleic acid” refers to any compound and/or substance that is or can be incorporated into an oligonucleotide chain. In some embodiments e.g., as set forth herein, a nucleic acid is a compound and/or substance that is or can be incorporated into an oligonucleotide chain via a phosphodiester linkage. As will be clear from context, in some embodiments e.g., as set forth herein, the term nucleic acid refers to an individual nucleic acid residue (e.g., a nucleotide and/or nucleoside), and in some embodiments e.g., as set forth herein refers to an polynucleotide chain comprising a plurality of individual nucleic acid residues. A nucleic acid can be or include DNA, RNA, or a combinations thereof. A nucleic acid can include natural nucleic acid residues, nucleic acid analogs, and/or synthetic residues. In some embodiments e.g., as set forth herein, a nucleic acid includes natural nucleotides (e.g., adenosine, thymidine, guanosine, cytidine, uridine, deoxyadenosine, deoxythymidine, deoxy guanosine, and deoxycytidine). In some embodiments e.g., as set forth herein, a nucleic acid is or includes of one or more nucleotide analogs (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguano sine, 8-oxoadenosine, 8-oxoguanosine, 0(6)-methylguanine, 2-thiocytidine, methylated bases, intercalated bases, and combinations thereof).

In some embodiments e.g., as set forth herein, a nucleic acid has a nucleotide sequence that encodes a functional gene product such as an RNA or protein. In some embodiments e.g., as set forth herein, a nucleic acid includes one or more introns. In some embodiments e.g., as set forth herein, a nucleic acid includes one or more genes. In some embodiments e.g., as set forth herein, nucleic acids are prepared by one or more of isolation from a natural source, enzymatic synthesis by polymerization based on a complementary template (in vivo or in vitro), reproduction in a recombinant cell or system, and chemical synthesis.

In some embodiments e.g., as set forth herein, a nucleic acid analog differs from a nucleic acid in that it does not utilize a phosphodiester backbone. For example, in some embodiments e.g., as set forth herein, a nucleic acid can include one or more peptide nucleic acids, which are known in the art and have peptide bonds instead of phosphodiester bonds in the backbone. Alternatively or additionally, in some embodiments e.g., as set forth herein, a nucleic acid has one or more phosphorothioate and/or 5′-N-phosphoramidite linkages rather than phosphodiester bonds. In some embodiments e.g., as set forth herein, a nucleic acid comprises one or more modified sugars (e.g., 2′-fluororibose, ribose, 2′-deoxyribose, arabinose, and hexose) as compared with those in natural nucleic acids.

In some embodiments, e.g., as set forth herein, a nucleic acid is or includes at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues. In some embodiments, e.g., as set forth herein, a nucleic acid is partly or wholly single stranded, or partly or wholly double stranded.

Nucleic acid detection assay: As used herein, the term “nucleic acid detection assay” refers to any method of determining the nucleotide composition of a nucleic acid of interest. Nucleic acid detection assays include but are not limited to, DNA sequencing methods (e.g., next generation sequencing methods, third generation sequencing methods, e.g., nanopore sequencing), polymerase chain reaction-based methods, probe hybridization methods, ligase chain reaction, etc.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context).

Oligonucleotide primer: As used herein, the term oligonucleotide primer, or primer, refers to a nucleic acid molecule used, capable of being used, or for use in, generating amplicons from a template nucleic acid molecule. Under transcription-permissive conditions (e.g., in the presence of nucleotides and a DNA polymerase, and at a suitable temperature and pH), an oligonucleotide primer can provide a point of initiation of transcription from a template to which the oligonucleotide primer hybridizes. Typically, an oligonucleotide primer is a single-stranded nucleic acid between 5 and 200 nucleotides in length. Those of skill in the art will appreciate that optimal primer length for generating amplicons from a template nucleic acid molecule can vary with conditions including temperature parameters, primer composition, and transcription or amplification method. A pair of oligonucleotide primers, as used herein, refers to a set of two oligonucleotide primers that are respectively complementary to a first strand and a second strand of a template double-stranded nucleic acid molecule. First and second members of a pair of oligonucleotide primers may be referred to as a “forward” oligonucleotide primer and a “reverse” oligonucleotide primer, respectively, with respect to a template nucleic acid strand, in that the forward oligonucleotide primer is capable of hybridizing with a nucleic acid strand complementary to the template nucleic acid strand, the reverse oligonucleotide primer is capable of hybridizing with the template nucleic acid strand, and the position of the forward oligonucleotide primer with respect to the template nucleic acid strand is 5′ of the position of the reverse oligonucleotide primer sequence with respect to the template nucleic acid strand. It will be understood by those of skill in the art that the identification of a first and second oligonucleotide primer as forward and reverse oligonucleotide primers, respectively, is arbitrary inasmuch as these identifiers depend upon whether a given nucleic acid strand or its complement is utilized as a template nucleic acid molecule.

Polyposis syndromes: The terms “polyposis” and “polyposis syndrome”, as used herein, refer to hereditary conditions that include, but are not limited to, familial adenomatous polyposis (FAP), hereditary nonpolyposis colorectal cancer (HNPCC)/Lynch syndrome, Gardner syndrome, Turcot syndrome, MUTYH polyposis, Peutz-Jeghers syndrome, Cowden disease, familial juvenile polyposis, and hyperplastic polyposis. In certain embodiments, polyposis includes serrated polyposis syndrome. Serrated polyposis is classified by a subject having 5 or more serrated polyps proximal to the sigmoid colon with two or more at least 10 mm in size, having a serrated polyp proximal to the sigmoid colon in the context of a family history of serrated polyposis, and/or having 20 or more serrated polyps throughout the colon.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

Probe: As used herein, the terms “probe”, “capture probe”, or “bait” refer to a single- or double-stranded nucleic acid molecule that is capable of hybridizing with a complementary target and includes a detectable moiety. In certain embodiments, e.g., as set forth herein, a probe is a restriction digest product or is a synthetically produced nucleic acid, e.g., a nucleic acid produced by recombination or amplification. In some instances, e.g., as set forth herein, a probe is a capture probe useful in detection, identification, and/or isolation of a target sequence, such as a gene sequence. In various instances, e.g., as set forth herein, a detectable moiety of probe can be, e.g., an enzyme (e.g., ELISA, as well as enzyme-based histochemical assays), fluorescent moiety, radioactive moiety, or moiety associated with a luminescence signal.

Promoter: As used herein, a “promoter” can refer to a DNA regulatory region that directly or indirectly (e.g., through promoter-bound proteins or substances) associates with an RNA polymerase and participates in initiation of transcription of a coding sequence.

Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.

Risk: As used herein with respect to a disease, disorder, or condition, the term “risk” refers to the qualitative of quantitative probability (whether expressed as a percentage or otherwise) that a particular individual will develop the disease, disorder, or condition. In some embodiments, e.g., as set forth herein, risk is expressed as a percentage. In some embodiments, e.g., as set forth herein, a risk is a qualitative of quantitative probability that is equal to or greater than 0, 1, 2, 3, 4, 5, 6, 7, 8, 9, 10, 20, 30, 40, 50, 60, 70, 80, 90, or 100%. In some embodiments, e.g., as set forth herein, risk is expressed as a qualitative or quantitative level of risk relative to a reference risk or level or the risk of the same outcome attributed to a reference. In some embodiments, e.g., as set forth herein, relative risk is increased or decreased in comparison to the reference sample by a factor of 1.1, 1.2, 1.3, 1.4, 1.5, 1.6, 1.7,. 1.8, 1.9, 2, 3, 4, 5, 6, 7, 8, 9, 10, or more.

Room Temperature: As used herein, room temperature refers to the ambient temperature, for example, in a laboratory in which the methods herein are conducted. In certain embodiments, room temperature is about 20° C. (e.g., from about 19° C. to about 21° C., from about 17° C. to about 23° C.).

Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as amplification or reverse transcription of nucleic acids, isolation and/or purification of certain components, etc.

In certain instances, e.g., as set forth herein, a processed sample can be a DNA sample that has been amplified (e.g., pre-amplified). Thus, in various instances, e.g., as set forth herein, an identified sample can refer to a primary form of the sample or to a processed form of the sample. In some instances, e.g., as set forth herein, a sample that is enzyme-digested DNA can refer to primary enzyme-digested DNA (the immediate product of enzyme digestion) or a further processed sample such as enzyme-digested DNA that has been subject to an amplification step (e.g., an intermediate amplification step, e.g., pre-amplification) and/or to a filtering step, purification step, or step that modifies the sample to facilitate a further step, e.g., in a process of determining methylation status (e.g., methylation status of a primary sample of DNA and/or of DNA as it existed in its original source context) or mutation status.

Screening: As used herein, the term “screening” refers to any method, technique, process, or undertaking intended to generate diagnostic information and/or prognostic information. Accordingly, those of skill in the art will appreciate that the term screening encompasses method, technique, process, or undertaking that determines whether an individual has, is likely to have or develop, or is at risk of having or developing a disease, disorder, or condition, e.g., colorectal cancer, advanced adenoma.

Single Nucleotide Polymorphism (SNP): As used herein, the term “single nucleotide polymorphism” or “SNP” refers to a particular base position in the genome where alternative bases are known to distinguish one allele from another. In some embodiments, one or a few SNPs and/or CNPs is/are sufficient to distinguish complex genetic variants from one another so that, for analytical purposes, one or a set of SNPs and/or CNPs may be considered to be characteristic of a particular variant, trait, cell type, individual, species, etc, or set thereof. In some embodiments, one or a set of SNPs and/or CNPs may be considered to define a particular variant, trait, cell type, individual, species, etc, or set thereof.

Solid Tumor: As used herein, the term “solid tumor” refers to an abnormal mass of tissue including cancer cells. In various embodiments, e.g., as set forth herein, a solid tumor is or includes an abnormal mass of tissue that does not contain cysts or liquid areas. In some embodiments, e.g., as set forth herein, a solid tumor can be benign; in some embodiments, a solid tumor can be malignant. Examples of solid tumors include carcinomas, lymphomas, and sarcomas. In some embodiments, e.g., as set forth herein, solid tumors can be or include adrenal, bile duct, bladder, bone, brain, breast, cervix, colon, endometrium, esophagum, eye, gall bladder, gastrointestinal tract, kidney, larynx, liver, lung, nasal cavity, nasopharynx, oral cavity, ovary, penis, pituitary, prostate, retina, salivary gland, skin, small intestine, stomach, testis, thymus, thyroid, uterine, vaginal, and/or vulval tumors.

Stage of cancer: As used herein, the term “stage of cancer” refers to a qualitative or quantitative assessment of the level of advancement of a cancer. In some embodiments, e.g., as set forth herein, criteria used to determine the stage of a cancer can include, but are not limited to, one or more of where the cancer is located in a body, tumor size, whether the cancer has spread to lymph nodes, whether the cancer has spread to one or more different parts of the body, etc. In some embodiments, e.g., as set forth herein, cancer can be staged using the so-called TNM System, according to which T refers to the size and extent of the main tumor, usually called the primary tumor; N refers to the number of nearby lymph nodes that have cancer; and M refers to whether the cancer has metastasized. In some embodiments, e.g., as set forth herein, a cancer can be referred to as Stage 0 (abnormal cells are present but have not spread to nearby tissue, also called carcinoma in situ, or CIS; CIS is not cancer, but it can become cancer), Stage I-III (cancer is present; the higher the number, the larger the tumor and the more it has spread into nearby tissues), or Stage IV (the cancer has spread to distant parts of the body). In some embodiments, e.g., as set forth herein, a cancer can be assigned to a stage selected from the group consisting of: in situ (abnormal cells are present but have not spread to nearby tissue); localized (cancer is limited to the place where it started, with no sign that it has spread); regional (cancer has spread to nearby lymph nodes, tissues, or organs): distant (cancer has spread to distant parts of the body); and unknown (there is not enough information to identify cancer stage).

Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status (e.g., a methylation status) associated with, development of the disease, disorder, or condition. In some embodiments, e.g., as set forth herein, a risk of developing a disease, disorder, and/or condition is a population-based risk (e.g., family members of individuals suffering from the disease, disorder, or condition). In some embodiments, a subject who is susceptible to a disease, disorder or condition is may be suspected of having and/or developing the disease, disorder, or condition.

Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to or suspected of having a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”

Upstream: As used herein, the term “upstream” means a first DNA region is closer, relative to a second DNA region, to the N-terminus of a nucleic acid that includes the first DNA region and the second DNA region.

Unmethylated: As used herein, the terms “unmethylated” and “non-methylated” are used interchangeably and mean that an identified DNA region includes no methylated nucleotides.

Variant: As used herein, the term “variant” refers to an entity that shows significant structural identity with a reference entity but differs structurally from the reference entity in the presence, absence, or level of one or more chemical moieties as compared with the reference entity. In some embodiments, e.g., as set forth herein, a variant also differs functionally from its reference entity. In general, whether a particular entity is properly considered to be a “variant” of a reference entity is based on its degree of structural identity with the reference entity. A variant can be a molecule comparable, but not identical to, a reference. For example, a variant nucleic acid can differ from a reference nucleic acid at one or more differences in nucleotide sequence. In some embodiments, e.g., as set forth herein, a variant nucleic acid shows an overall sequence identity with a reference nucleic acid that is at least 85%, 86%, 87%, 88%, 89%, 90%, 91%, 92%, 93%, 94%, 95%, 96%, 97%, or 99%. In many embodiments, e.g., as set forth herein, a nucleic acid of interest is considered to be a “variant” of a reference nucleic acid if the nucleic acid of interest has a sequence that is identical to that of the reference but for a small number of sequence alterations at particular positions. In some embodiments, e.g., as set forth herein, a variant has 10, 9, 8, 7, 6, 5, 4, 3, 2, or 1 substituted residues as compared with a reference. In some embodiments, e.g., as set forth herein, a variant has not more than 5, 4, 3, 2, or 1 residue additions, substitutions, or deletions as compared with the reference. In various embodiments, e.g., as set forth herein, the number of additions, substitutions, or deletions is fewer than about 25, about 20, about 19, about 18, about 17, about 16, about 15, about 14, about 13, about 10, about 9, about 8, about 7, about 6, and commonly are fewer than about 5, about 4, about 3, or about 2 residues.

### DETAILED DESCRIPTION

It is contemplated that systems, devices, methods, and processes of the claimed invention encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems of the present invention that consist essentially of, or consist of, the recited components, and that there are processes and methods according to the present invention that consist essentially of, or consist of, the recited processing steps. It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents mentioned herein are incorporated by reference. Where there is any discrepancy in the meaning of a particular term, the meaning provided in the Definition section above is controlling.

Headers are provided for the convenience of the reader—the presence and/or placement of a header is not intended to limit the scope of the subject matter described herein.

### Advanced Adenomas

In certain embodiments, methods and compositions presented herein are useful for screening for advanced adenomas. Advanced adenomas include, without limitation: neoplastic adenomatous growth in colon and/or in rectum, adenomas located in the proximal part of the colon, adenomas located in the distal part of the colon and/or rectum, adenomas of low grade dysplasia, adenomas of high grade dysplasia, neoplastic growth(s) of colorectum tissue that shows signs of high grade dysplasia of any size, neoplastic growth(s) of colorectum tissue having a size greater than or equal to 10mm of any histology and/or dysplasia grade, neoplastic growth(s) of colorectum tissue with villious histological type of any type of dysplasia and any size, and colorectum tissue having a serrated histological type with any dysplasia grade and/or size.

### Colorectal Cancers

In certain embodiments, methods and compositions of the present disclosure are useful for screening for colorectal cancer (e.g., in a subject susceptible to or at risk of developing colorectal cancer). Colorectal cancers include, without limitation, colon cancer, rectal cancer, and combinations thereof. Colorectal cancers include metastatic colorectal cancers and non-metastatic colorectal cancers. Colorectal cancers include cancer located in the proximal part of the colon cancer and cancer located in the distal part of the colon.

Colorectal cancers include colorectal cancers at any of the various possible stages known in the art, including, e.g., Stage I, Stage II, Stage III, and Stage IV colorectal cancers (e.g., stages 0, I, IIA, IIB, IIC, IIIA, IIIB, IIIC, IVA, IVB, and IVC). Colorectal cancers include all stages of the Tumor/Node/Metastasis (TNM) staging system. With respect to colorectal cancer, T can refer to whether the tumor grown into the wall of the colon or rectum, and if so by how many layers; N can refer to whether the tumor has spread to lymph nodes, and if so how many lymph nodes and where they are located; and M can refer to whether the cancer has spread to other parts of the body, and if so which parts and to what extent. Particular stages of T, N, and M are known in the art. T stages can include TX, T0, Tis, T1, T2, T3, T4a, and T4b; N stages can include NX, N0, N1a, N1b, N1c, N2a, and N2b; M stages can include MO, M1a, and M1b. Moreover, grades of colorectal cancer can include GX, G1, G2, G3, and G4. Various means of staging cancer, and colorectal cancer in particular, are well known in the art summarized, e.g., on the world wide web at cancer.net/cancer-types/colorectal-cancer/stages.

In certain instances, the present disclosure includes screening of early stage colorectal cancer. Early stage colorectal cancers can include, e.g., colorectal cancers localized within a subject, e.g., in that they have not yet spread to lymph nodes of the subject, e.g., lymph nodes near to the cancer (stage N0), and have not spread to distant sites (stage M0). Early stage cancers include colorectal cancers corresponding to, e.g., Stages 0 to II C.

Thus, colorectal cancers of the present disclosure include, among other things, pre-malignant colorectal cancer and malignant colorectal cancer. Methods and compositions of the present disclosure are useful for screening of colorectal cancer in all of its forms and stages, including without limitation those named herein or otherwise known in the art, as well as all subsets thereof. Accordingly, the person of skill in art will appreciate that all references to colorectal cancer provided here include, without limitation, colorectal cancer in all of its forms and stages, including without limitation those named herein or otherwise known in the art, as well as all subsets thereof.

### Subjects and Samples

A sample analyzed using methods and compositions provided herein can be any biological sample and/or any sample including nucleic acids. In various particular embodiments, a sample analyzed using methods and compositions provided herein can be a sample from a mammal. In various particular embodiments, a sample analyzed using methods and compositions provided herein can be a sample from a human subject. In various particular embodiments, a sample analyzed using methods and compositions provided herein can be a sample form a mouse, rat, pig, horse, chicken, or cow.

In various instances, a human subject is a subject diagnosed or seeking diagnosis as having, diagnosed as or seeking diagnosis as at risk of having, and/or diagnosed as or seeking diagnosis as at immediate risk of having a disease related to aberrant methylation and/or a mutation in one or more loci of the genome (e.g., cancer). In various instances, a human subject is a subjected identified as a subject in need of screening for a disease or condition (e.g., cancer, e.g., colorectal cancer, advanced adenoma). In certain instances, a human subject is a subject identified as in need of screening by a medical practitioner (e.g., colorectal cancer screening). In various instances, a human subject is identified as in need of screening due to age, e.g., due to an age equal to or greater than 40 years, e.g., an age equal to or greater than 49, 45, 50, 55, 60, 65, 70, 75, 80, 85, or 90 years, though in some instances a subject 18 years old or older may be identified as at risk, susceptible to, and/or in need of screening for a disease, disorder, or condition (e.g., cancer, e.g., colorectal cancer, advanced adenoma). In various instances, a human subject is identified as being high risk and/or in need of screening for a neoplasm (e.g., colorectal cancer, advanced adenoma) based on, without limitation, familial history, prior diagnoses, and/or an evaluation by a medical practitioner. In various instances, a human subject is a subject not diagnosed as having, not at risk of having, not at immediate risk of having, not diagnosed as having, and/or not seeking diagnosis for a disease, disorder, and/or condition (e.g., a cancer such as a colorectal cancer, or any combination thereof).

A sample from a subject, e.g., a human or other mammalian subject, can be a sample of, e.g., blood, blood component (e.g., plasma, buffy coat), cfDNA (cell free DNA), ctDNA (circulating tumor DNA), stool, or tissue (e.g., advanced adenoma and/or colorectal tissue). In some particular embodiments, a sample is an excretion or bodily fluid of a subject (e.g., stool, blood, plasma, lymph, or urine of a subject) or a tissue sample of a colorectal neoplasm, such as a colonic polyp, an advanced adenoma, and/or colorectal cancer. A sample from a subject can be a cell or tissue sample, e.g., a cell or tissue sample that is of a cancer or includes cancer cells, e.g., of a tumor or of a metastatic tissue. For example, the sample may include colorectal cells, polyp cells, or glandular cells. In various embodiments, a sample from a subject, e.g., a human or other mammalian subject, can be obtained by biopsy (e.g., colonoscopy resection, fine needle aspiration or tissue biopsy) or surgery.

In various particular embodiments, a sample is a sample of cell-free DNA (cfDNA). cfDNA is typically found in biological fluids (e.g., plasma, serum, or urine) in short, double-stranded fragments. The concentration of cfDNA is typically low, but can significantly increase under particular conditions, including without limitation pregnancy, autoimmune disorder, myocardial infraction, and cancer. Circulating tumor DNA (ctDNA) is the component of circulating DNA specifically derived from cancer cells. ctDNA can be present in human fluids. For example in some instances, ctDNA can be found bound to and/or associated with leukocytes and erythrocytes. In some instances, ctDNA can be found not bound to and/or associated with leukocytes and erythrocytes. Various tests for detection of tumor-derived cfDNA are based on detection of genetic or epigenetic modifications that are characteristic of cancer (e.g., of a relevant cancer). Genetic or epigenetic modifications characteristic of cancer can include, without limitation, oncogenic or cancer-associated mutations in tumor-suppressor genes, activated oncogenes, hypermethylation, and/or chromosomal disorders. Detection of genetic or epigenetic modifications characteristic of cancer or pre-cancer can confirm that detected cfDNA is ctDNA.

cfDNA and ctDNA provide a real-time or nearly real-time metric of the methylation status of a source tissue. cfDNA and ctDNA have a half-life in blood of about 2 hours, such that a sample taken at a given time provides a relatively timely reflection of the status of a source tissue.

Various methods of isolating nucleic acids from a sample (e.g., of isolating cfDNA from blood or plasma) are known in the art. Nucleic acids can be isolated, e.g., without limitation, standard DNA purification techniques, by direct gene capture (e.g., by clarification of a sample to remove assay-inhibiting agents and capturing a target nucleic acid, if present, from the clarified sample with a capture agent to produce a capture complex, and isolating the capture complex to recover the target nucleic acid).

In certain embodiments, a sample may have a required minimum amount of DNA (e.g., cfDNA, gDNA) (e.g.,DNA fragments) for later determining a methylation status. For example, in certain embodiments, a sample may be required to have at least 5 ng, at least 9 ng, at least 10 ng, at least 20 ng (or more) DNA. In certain embodiments, a sample may be required to have from from 5 ng to 25 ng (e.g., 10 ng to 20 ng) of DNA. In certain embodiments, at least 1mL (e.g., at least 2mL, at least 3mL, at least 4mL, at least 5 ml or more) of human plasma is used for cfDNA extraction. In certain embodiments, about 4 ml to about 5 ml of human plasma is used (e.g., from about 4 ml to about 5 ml, about 3 ml to about 6 ml).

### Methods of Measuring Methylation Status

Methylation status can be measured by a variety of methods known in the art and/or by methods provided in this specification.

In certain embodiments, the processing steps involve fragmenting or shearing DNA of the sample. For example, genomic DNA (e.g., gDNA) obtained from a cell, tissue, or other source may require fragmentation prior to sequencing. In certain embodiments, DNA may be fragmented prior to measurement of methylation status using a physical method (e.g., using an ultra-sonicator, a nebulizer technique, hydrodynamic shearing, etc.). In certain embodiments, DNA may be fragmented using an enzymatic method (e.g., using an endonuclease or a transposase). Certain samples, e.g., cfDNA samples, may not require fragmentation. Certain technologies may require DNA fragments of about 100-1000bp range. DNA fragments of about 10 kb or longer (e.g., at least 1 kb, at least 2 kb, at least 3 kb, at least 4 kb, at least 5 kb, at least 6 kb, at least 7 kb, at least 8 kb, at least 9 kb, at least 10 kb or longer) are suitable for long read sequencing technologies (e.g., third generation sequencing, e.g., nanopore sequencing).

Certain particular assays for methylation utilize a bisulfite reagent (e.g., hydrogen sulfite ions) or enzymatic conversion reagents (e.g., Tet methylcytosine dioxygenase 2).

Bisulfite reagents can include, among other things, bisulfite, disulfite, hydrogen sulfite, sodium metabisulphite, or combinations thereof, which reagents can be useful in distinguishing methylated and unmethylated nucleic acids. Bisulfite interacts differently with cytosine and 5-methylcytosine. In typical bisulfite-based methods, contacting of DNA (e.g., single stranded DNA, double stranded DNA) with bisulfite deaminates (e.g., converts) unmethylated cytosine to uracil, while methylated cytosine remains unaffected. Methylated cytosines, but not unmethylated cytosines, are selectively retained. Thus, in a bisulfite processed sample, uracil residues stand in place of, and thus provide an identifying signal for, unmethylated cytosine residues, while remaining (methylated) cytosine residues thus provide an identifying signal for methylated cytosine residues. Bisulfite processed samples can be analyzed, e.g., by next generation sequencing (NGS) or other methods disclosed herein.

Enzymatic conversion reagents can include Tet methylcytosine dioxygenase 2 (TET2). TET2 oxidizes 5-methylcytosine and thus protects it from the consecutive deamination by APOBEC. APOBEC deaminates unmethylated cytosine to uracil, while oxidized 5-methylcytosine remains unaffected. Thus, in a TET2 processed sample, uracil residues stand in place of, and thus provide an identifying signal for, unmethylated cytosine residues, while remaining (methylated) cytosine residues thus provide an identifying signal for methylated cytosine residues. TET2 processed samples can be analyzed, e.g., by next generation sequencing (NGS). In certain embodiments, APOBEC refers to a member (or plurality of members) of the Apolipoprotein B mRNA Editing Catalytic Polypeptide-like (APOBEC) family. In certain embodiments, APOBEC may refer to APOBEC-1, APOBEC-2, APOBEC-3A, APOBEC-3B, APOBEC-3C, APOBEC-3D, APOBEC-3E, APOBEC-3F, APOBEC-3G. APOBEC-3H, APOBEC-4, and/or Activation-induced (cytidine) deaminase (AID).

Methods of measuring methylation status can include, without limitation, massively parallel sequencing (e.g., next-generation sequencing, e.g., third generation sequencing) to determine methylation state, e.g., sequencing by-synthesis, real-time (e.g., single-molecule) sequencing, bead emulsion sequencing, nanopore sequencing, or other sequencing techniques known in the art. In some embodiments, a method of measuring methylation status can include whole-genome sequencing, e.g., measuring whole genome methylation status from bisulfite or enzymatically treated material with base-pair resolution.

In some embodiments, the pre-selection (capture) (e.g., enrichment) of regions of interest (e.g., DMRs) can be done by complementary in vitro synthesized oligonucleotide sequences (e.g., capture baits/probes). Capture probes (e.g., oligonucleotide capture probes, oligonucleotide capture baits) are useful in targeted sequencing (e.g., NGS) techniques to enrich for particular regions of interest in an oligonucleotide (e.g., DNA) sequence. For example, enrichment of target regions is useful when sequences of particular pre-determined regions of DNA are sequenced. In certain embodiments, capture probes are about 10bp to 1000bp long (e.g., about 10bp to about 200bp long) (e.g., about 120bp long). In certain embodiments, one or more capture probes are targeted to capture a region of interest (e.g., a genomic marker) corresponding to one or more methylation loci (e.g., methylation loci comprising at least a portion of one or more DMRs). In certain embodiments, capture probes are targeted to methylation loci that are hypomethylated or hypermethylated. For example, a capture probe may be targeted to a particular methylation loci. However, if fragments of DNA corresponding to a methylation loci are converted (e.g., bisulfite or enzymatic converted) prior to enrichment using a capture probe, the sequence of the converted DNA fragments will change as described herein due to particular cytosine residues being unmethylated. Therefore, targeting an unconverted DNA region may result in some mismatches if cytosines are hypomethylated. Though capture probe-target sequence hybridization may tolerate some mismatches, a second probe may be required to enrich for DNA regions which are hypomethylated.

In certain embodiments, capture probes are evaluated (e.g., prior to sequencing) for their ability to target multiple regions of the genome of interest. For example, when designing a capture probe to target a particular region of interest (e.g., a DMR), the ability for a capture probe to target multiple regions of the genome may be considered. Mismatches in pairing (e.g., non-Watson-Crick pairing) allow for capture probes to hybridize to other, unintended regions of a genome. In addition, a particular target sequence may be repeated elsewhere in a genome. Repeat sequences are common for sequences that are highly repetitive. In certain embodiments, capture probes are designed such that they only target a few similar regions of the genome. In certain embodiments, capture probes may hybridize to 500 or fewer, 100 or fewer, 50 or fewer, 10 or fewer, 5 or fewer similar regions in a genome. In certain embodiments, a similar region to the target of region of interest is calculated using a 24bp window moving around a genome and matching the region of the window to a reference sequence according to sequence order similarity. Other size windows and/or techniques may be used.

For example, hybrid-capture of one or more DNA fragments (e.g., ctDNA, fragmented gDNA) may be performed using capture probes targeted to predetermined regions of interested of a genome. In certain embodiments, capture probes target at least 2 (e.g, at least 3, 4, 5, 6, 7, 8, 9, 10, 11, 12, 13, 14, 15, 16, 17, 18, 19, 20, 21, 22, 23, 24, 25, 50, 75, 100, 150, or more) predetermined regions of interest (e.g., genomic markers, e.g., DMRs). In certain embodiments, the capture probes overlap. In certain embodiments, the overlapping probes overlap at least 10%, at least 20%, at least 30%, at least 40%, at least 50%, at least 60% or more.

In certain embodiments, the capture probes are nucleic acid probes (e.g., DNA probes, RNA probes). In some embodiments, a method may also include identifying mutated regions (e.g., individual nucleotide bases) using targeted sequencing e.g., determining the presence of a mutation in one or more pre-selected genomic locations (e.g., a genomic marker, e.g., a mutation marker). In certain embodiments, mutations may also be identified from bisulfite or enzymatically treated DNA with base-pair resolution.

In some embodiments, a sequencing library may be prepared using converted (e.g., enzyme converted) oligonucleotide fragments (e.g., cfDNA, gDNA fragments, synthetic nucleotide sequences, etc.) according to, e.g., an Illumina protocol, an Accel-NGS® Methyl-Seq DNA Library Kit (Swift Bioscience) protocol, a transpose-based Nextera XT protocol, or the like. In some embodiments, the oligonucleotide fragments are DNA fragments which have been converted (e.g., enzyme converted). In certain embodiments, DNA fragments used in preparation of a sequencing library may be single stranded DNA fragments or double stranded DNA fragments. In certain embodiments, a library may be prepared by attaching adapters to DNA fragments. Adapters contain short sequences (e.g., oligonucleotide sequences) that allow oligonucleotide fragments of a library (e.g., a DNA library) to bind to and generate clusters on a flow cell used in, for example, next generation sequencing (NGS) (e.g., third generation sequencing). Adapters may be ligated to library fragments prior to NGS. In certain embodiments, a ligase enzyme covalently links the adapter and library fragments. In certain embodiments, adapters are attached to either one or both of the 5′ and 3′ ends of converted DNA fragments. In certain embodiments, the attaching step is performed such that at least 40%, at least 50%, at least 60%, at least 70% of the converted DNA fragments are attached to adapter. In certain embodiments, the attaching step is performed such that at least 40%, at least 50%, at least 60%, at least 70% of the converted DNA fragments have an adapter attached at both the 5′ and 3′ ends

In certain embodiments, adapters used herein contain a sequence of oligonucleotides that aid in sample identification. For example, in certain embodiments, adapters include a sample index. A sample index is a short sequence (e.g., 8 bases to 10 bases, 5 bases to 12 bases) (e.g., at least 4, at least 5, at least 6, at least 7, at least 8 bases or more) (fewer than 50 bases, fewer than 40 bases, fewer than 30 bases) of nucleic acids (e.g., DNA, RNA) that serve as sample identifiers and allow for, among other things, multiplexing and/or pooling of multiple samples in a single sequencing run and/or on a flow cell (e.g., used in a NGS technique). In certain embodiments, an adapter at a 5′ end, a 3′ end, or both of a converted single stranded DNA fragment includes a sample index. In certain embodiments, an adapter sequence may include a molecular barcode. A molecular barcode may serve as a unique molecular identifier to identify a target molecule during, for example, DNA sequencing. In certain embodiments, DNA barcodes may be randomly generated. In certain embodiments, DNA barcodes may be predetermined or predesigned. In certain embodiments, the DNA barcodes are different on each DNA fragment. In certain embodiments, the DNA barcodes may be the same for two single stranded DNA fragments that are not complementary to one another (e.g., in a Watson-Crick pair with each other) in the biological sample. In certain embodiments, DNA fragments may be amplified (e.g., using PCR) after ligation of adapters to DNA fragments. In certain embodiments, at least 40% (e.g., at least at least 50%, at least 60%, at least 70%) of the converted DNA fragments have an adapter attached at both the 5′ and 3′ ends.

Those of skill in the art will appreciate that in embodiments in which a plurality of methylation loci (e.g., a plurality of DMRs) are analyzed for methylation status in a method of screening for colorectal cancer provided herein, methylation status of each methylation locus can be measured or represented in any of a variety of forms, and the methylation statuses of a plurality of methylation loci (preferably each measured and/or represented in a same, similar, or comparable manner) be together or cumulatively analyzed or represented in any of a variety of forms. In various embodiments, methylation status of each methylation locus can be measured as methylation portion. In various embodiments, methylation status of each methylation locus can be represented as the percentage value of methylated reads from total sequencing reads compared against reference sample. In various embodiments, methylation status of each methylation locus can be represented as a qualitative comparison to a reference, e.g., by identification of each methylation locus as hypermethylated or hypomethylated.

In some embodiments in which a single methylation locus is analyzed, hypermethylation of the single methylation locus constitutes a diagnosis that a subject is suffering from or possibly suffering from a condition (e.g., cancer) (e.g., advanced adenoma, colorectal cancer), while absence of hypermethylation of the single methylation locus constitutes a diagnosis that the subject is likely not suffering from a condition. In some embodiments, hypermethylation of a single methylation locus (e.g., a single DMR) of a plurality of analyzed methylation loci constitutes a diagnosis that a subject is suffering from or possibly suffering from the condition, while the absence of hypermethylation at any methylation locus of a plurality of analyzed methylation loci constitutes a diagnosis that a subject is likely not suffering from the condition. In some embodiments, hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is suffering from or possibly suffering from the condition, while the absence of hypermethylation of a determined percentage (e.g., a predetermined percentage) of methylation loci (e.g., at least 10% (e.g., at least 10%, at least 20%, at least 30%, at least 40%, at least 50%, at least 60%, at least 70%, at least 80%, at least 90%, or 100%)) of a plurality of analyzed methylation loci constitutes a diagnosis that a subject is not likely suffering from the condition. In some embodiments, hypermethylation of a determined number (e.g., a predetermined number) of methylation loci (e.g., at least 1, 2, 3, 4, 5, 6, 7, 8, 9, 10, 11, 12, 13, 14, 15, 16, 17, 18, 19, 20, 21, 22, 23, 24, 25, 26, 50, 100, 150, or more DMRs) of a plurality of analyzed methylation loci (e.g 2, 3, 4, 5, 6, 7, 8, 9, 10, 11, 12, 13, 14, 15, 16, 17, 18, 19, 20, 21, 22, 23, 24, 25, 26, 50, 100, 150, or more DMRs) constitutes a diagnosis that a subject is suffering from or possibly suffering from the condition, while the absence of hypermethylation of a determined number (e.g., a predetermined number) of methylation loci (e.g., at least 1, 2, 3, 4, 5, 6, 7, 8, 9, 10, 11, 12, 13, 14, 15, 16, 17, 18, 19, 20, 21, 22, 23, 24, 25, 26, 50, 100, 150, or more DMRs) of a plurality of analyzed methylation loci (e.g., 2, 3, 4, 5, 6, 7, 8, 9, 10, 11, 12, 13, 14, 15, 16, 17, 18, 19, 20, 21, 22, 23, 24, 25, 26, 50, 100, 150, or more DMRs) constitutes a diagnosis that a subject is not likely suffering from the condition.

In some embodiments, methylation status of a plurality of methylation loci (e.g., a plurality of DMRs) is measured qualitatively or quantitatively and the measurement for each of the plurality of methylation loci are combined to provide a diagnosis. In some embodiments, the quantitatively measured methylation status of each of a plurality of methylation loci is individually weighted, and weighted values are combined to provide a single value that can be comparative to a reference in order to provide a diagnosis.

In some embodiments, methylation status may include determination of methylated and/or unmethylated reads mapped to a genomic region (e.g., a DMR). For example, when using particular sequencing technologies as disclosed herein, sequence reads are produced. A sequence read is an inferred sequence of base pairs (e.g., a probabilistic sequence) corresponding to all or part of a sequenced oligonucleotide (e.g., DNA) fragment (e.g., cfDNA fragments, gDNA fragments). In certain embodiments, sequence reads may be mapped (e.g., aligned) to a particular region of interest using a reference sequence (e.g., a bisulfite converted reference sequence) in order to determine if there are any alterations or variations in a read. Alterations may include methylation and/or mutations. A region of interest may include one or more genomic markers including a methylation marker (e.g., a DMR), a mutation marker, or other marker as disclosed herein.

For example, in the case of enzymatically treated DNA fragments, treatment converts unmethylated cytosines to uracils, while methylated cytosines are not converted to uracils. Accordingly, a sequence read produced for a DNA fragment that has methylated cytosines will be different from a sequence read produced for the same DNA fragment that does not have methylated cytosine. Methylation at sites where a cytosine nucleotide is followed by a guanine nucleotide (e.g., CpG sites) may be of particular interest.

### Quality Control Protocol

In certain embodiments, quality control steps may be implemented. Quality control steps are used to determine whether or not particular steps or processes were conducted within particular parameters. In certain embodiments, quality control steps may be used to determine the validity of results of a given analysis. In addition or alternatively, quality control steps may be used to determine sequenced data quality. For example, quality control steps may be used to determine read coverage of one or more regions of DNA. Quantitative metrics for quality control include, but are not limited to AT dropout rate, GC dropout rate, enzymatic conversion rate (e.g., enzymatic conversion efficiency), and the like. Failure to meet a threshold quality control condition (e.g., a minimum conversion rate, a maximum CG dropout rate, etc.) may indicate, for example, that one or more of the conversion steps were not performed within appropriate parameters.

For example, in the methods described herein, various steps of a conversion protocol may be optimized to decrease AT and/or GC dropout rate. As is understood by those of skill in the art, AT and GC dropout metrics indicate the degree of inadequate coverage of a particular target region based on its AT or GC content. In certain embodiments, samples having a low GC dropout rate is useful in identifying which samples were processed appropriately. For example, a GC dropout rate found to be less than 10%, less than 9%, less than 8%, less than 7%, less than 6%, less than 5%, less than 4%, or less may be useful in identifying appropriately processed samples.

### Artificial Spike-in Control

Control nucleic acid (e.g., DNA) molecules (e.g., “spike-in controls”) may be used to evaluate or estimate conversion efficiency of unmethylated and methylated cytosines to uracils. Control nucleic acid molecules may be used in sequencing methods involving conversion (e.g., enzymatic conversion) of DNA samples.

When DNA is subjected to conversion (e.g., enzymatic conversion) as described herein, conversion may be incomplete. That is, some number of unmethylated cytosines may not be converted to uracils. If the conversion is not complete such that unmethylated cytosines are not mostly converted, the unconverted unmethylated cytosines may be identified as methylated when the DNA is sequenced. Accordingly, in order to determine whether or not conversion is complete, a control DNA molecule may be subjected to conversion along with DNA fragments from a sample. In certain embodiments, sequencing the converted control DNA molecules (e.g., using a sequencing technique as described herein) generates a plurality of control sequence reads. Control sequence reads may be used to determine conversion rates of unmethylated and/or methylated cytosines to uracils.

In certain embodiments, spike-in controls (e.g., a control DNA molecule) are useful to include in each sample. In prior methods, it was presumed that conversion efficiencies remained relatively consistent between samples for a given run. However, the conversion rate of unmethylated cytosines to uracils in DNA fragments may vary significantly from on sample to another. For example, conversion efficiency may range from 10% to 110% within a single batch of processed samples. Note, there can be overconversion such that conversion efficiency can be greater than 100%, e.g., the conversion efficiency is 110% when 10% of the methylated cytosine gets converted. In certain embodiments, the conversion efficiency ranges from 30% to 110%. In other embodiments, the conversion efficiency ranges from 50% to 100%.

In certain embodiments, a control DNA molecule may be added to a sample after fragmentation and before conversion using e.g., enzymatic reagents. In certain embodiments, a plurality (e.g., two, three, four or more) control DNA sequences may be added to DNA fragments of a sample. A control DNA molecule may be a known sequence. For example, the sequence, number of methylated bases, and number of unmethylated bases of the control sequence had been determined prior to addition of the control DNA molecule to the sample. In certain embodiments, a control sequence may be a DNA sequence which is produced in vitro to contain artificially methylated or unmethylated nucleotides (e.g., methylated cytosines). In certain embodiments, a control sequence may be a DNA sequence which is produced to contain completely unmethylated DNA nucleotides.

A high conversion efficiency of the spike-in control sequence may be used to infer the conversion efficiency of a DNA fragments undergoing the same conversion process as a spike-in control. For example, deamination of at least at least 98% of unmethylated cytosines in the unmethylated spike-in control DNA sequence indicates that conversion efficiency is high and that a sample may pass a quality control assessment. In certain embodiments, at least 90%, at least 95%, at least 96%, at least 97%, at least 98%, at least 99% of unmethylated cytosines of a plurality of DNA fragments of a control DNA sequence are converted into uracils. A high conversion efficiency is important as it is ideal for all (or nearly all) of the unmethylated cytosines to be converted to uracils when subjecting DNA to bisulfite or enzymatic treatments. As described above, unconverted, unmethylated cytosines may serve as a source of noise in the data.

In addition, conversion of methylated cytosines to uracils is undesirable when DNA is treated using a conversion process. Conversion of methylated cyto sines of a spike-in control is indicative that methylated cytosines have been converted to uracils in a DNA sample subjected to the same treatment as the methylated spike-in control. Methylated cytosines in a methylated spike-in control should not convert to uracils. For the same reasons as described above, methylated cytosines being converted to uracils may result in misidentification of purportedly unmethylated cytosines during methylation analysis. In certain embodiments, at most 5%, at most 4%, at most 3%, at most 2% or at most 1% of methylated cytosines of a plurality of DNA fragments of a control DNA sequence are converted into uracils. For example, deamination of at most 2% of methylated cytosines in a methylated spike-in control DNA sequence indicates that conversion efficiency is high and that a sample may pass a quality control assessment.

### Adapters and Barcodes

In certain embodiments, adapters used herein contain a sequence of oligonucleotides that aid in sample identification. In certain embodiments, an adaptor is from 5 bases to 100 bases (e.g., less than 100 bases, less than 50 bases) (about 5 bases, about 10 bases, about 15 bases, about 20 bases, about 30 bases, about 34 bases, about 40 bases, about 50 bases). For example, in certain embodiments, adapters include a sample index. A sample index is a short sequence (e.g., about 5 to about 15 bases, e.g., about 8 bases to about 10 bases) of nucleic acids (e.g., DNA, RNA) that serve as sample identifiers and allow for, among other things, multiplexing and/or pooling of multiple samples in a single sequencing run and/or on a flow cell (e.g., used in a NGS technique, e.g., a third generation NGS technique). In certain embodiments, an adapter at a 5′ end, a 3′ end, or both of a converted single stranded DNA fragment includes a sample index. In certain embodiments, an adapter sequence may include a molecular barcode. A molecular barcode may serve as a unique molecular identifier to identify a target molecule during, for example, DNA sequencing. In certain embodiments, DNA barcodes may be randomly generated. In certain embodiments, DNA barcodes may be predetermined or predesigned. In certain embodiments, the DNA barcodes are different on each DNA fragment. In certain embodiments, the DNA barcodes may be the same for two single stranded DNA fragments that are not complementary to one another (e.g., in a Watson-Crick pair with each other) in the biological sample. In certain embodiments, DNA fragments may be amplified (e.g., using PCR) after ligation of adapters to DNA fragments. In certain embodiments, at least 40% (e.g., at least at least 50%, at least 60%, at least 70%) of the converted DNA fragments have an adapter attached at both the 5′ and 3′ ends.

### Identifying Mutations

In certain embodiments as disclosed herein, genomic mutations may be identified in one or more predetermined mutation biomarkers. In various embodiments, a mutation biomarker of the present disclosure is used for further detection (e.g., screening) and/or classification of a condition in addition to methylation biomarkers. In certain embodiments, information regarding a methylation status of one or more colorectal cancer biomarkers may be combined with a mutation biomarker in order to further classify the identified colorectal cancer. In addition or alternatively, mutation biomarkers may be used to determine or recommend (e.g., either for or against) a particular course of treatment for the identified disease and/or condition.

In certain embodiments, identifying genomic mutations may be performed using a sequencing technique as discussed herein (e.g., a third generation sequencing technique). In certain embodiments, oligonucleotides (e.g., cfDNA fragments, gDNA fragments) are sequenced to a read depth sufficient to detect a genomic mutation (e.g., in a mutation biomarker, in a tumor markers) at a frequency in a sample as low as 1.0%, 0.75%, 0.5%, 0.25%, 0.1%, 0.075%, 0.05%, 0.025%, 0.01%, or 0.005%.

Genomic mutations generally include any variation in nucleotide base pair sequences of DNA as is understood in the art. A mutation in a nucleic acid may, in some embodiments, include a single nucleotide variant, an inversion, a deletion, an insertion, a transversion, a translocation, a fusion, a truncation, an amplification, or a combination thereof, as compared to a reference DNA sequence.

Mutations may be identified using sequencing techniques discussed herein (e.g., a next generation sequencing technique, a third generation sequencing technique, nanopore sequencing. or the like). In certain embodiments as disclosed herein, mutations may be identified in converted (e.g., enzymatic converted) DNA fragments. In certain embodiments, mutations and methylated loci may be identified in parallel (e.g., simultaneously) using a single sequencing assay (e.g., an NGS assay, a third generation sequencing assay). In certain embodiments, one or more capture probes are targeted to capture and/or enrich for a region of interest of an oligonucleotide (e.g., DNA) sequence corresponding to one or more mutations markers.

In certain embodiments, mutation markers contain low GC content regions. Due to the low GC content, sufficient coverage of a region may not be obtained when sequencing a low GC content region using protocols adapted for high GC content regions. For example, targeted NGS sequencing (e.g., targeted bisulfite sequencing) of a low GC content region using only 1x tiling density of a target region may not provide sufficient coverage of a mutation region. Tiling (e.g., tiling density, tiling frequency) refers to a number of probes targeted to a region. Increased probe tiling density (e.g., through increasing the number of probes targeting a region) may be used in order to provide additional coverage for a region. For example, coverage of a low GC content region may be improved through increased tiling. Accordingly, increasing tiling density of a region to at least 2x tiling (e.g., 3x, 4x or more) may be beneficial in enhancing enrichment of a targeted region. For example, with 2x tiling, a region covered by a probe may be covered with at two probes which overlap with one another. In addition or alternatively, probes may be overlapped to permit enhanced coverage of a region. For example, probes may be overlapped by at least 10%, 20%, 30%, 40%, 50% or more. The amount which two probes overlap with one another may depend on desired tiling density, sequence of a targeted region, or other factors. For the avoidance of doubt, tiling and/or overlap of probes may also be changed over high GC content regions (e.g., methylation loci) as well.

### Kits

The present disclosure includes, among other things, kits including one or more compositions for use in performing the methods as provided herein, optionally in combination with instructions for use thereof in screening (e.g., screening for advanced adenoma, colorectal cancer, other cancers, or other diseases or conditions associated with an aberrant methylation and/or mutation status, e.g., neurodegenerative diseases, gastrointestinal disorders, and the like). In various embodiments, a kit for screening a diseases or conditions associated with an aberrant methylation status can include one or more oligonucleotide probes. In certain embodiments, the kit for screening optionally includes one or more enzymatic conversion reagents as disclosed herein. In certain embodiments, the kit for screening may include one or more adapters as described herein. In certain embodiments, the kit may include one or more reagents used in library preparation (e.g., as described herein). In certain embodiments, the kit may include software (e.g., for analyzing methylation status of DMRs, for analyzing one or more mutations).

### Preparing and Sequencing Samples

The present disclosure provides systems, methods, and apparatus for preparing biological samples for genetic sequencing (e.g., DNA sequencing, e.g., third generation generation sequencing). Moreover, the present disclosure provides various systems, methods, and apparatus that employ this sample preparation technology in the identification of biomarkers for detection of a disease or condition. In certain embodiments, the disease or condition is, for example, advanced adenoma, colorectal cancer, another cancer, or another disease or condition (e.g., neurodegenerative diseases, gastrointestinal disorders, and the like), particularly a disease or condition associated with an aberrant methylation status (e.g., hypermethylation or hypomethylation) and/or one or more genomic mutations (e.g., a single nucleotide variant, an inversion, a deletion, an insertion, a transversion, a translocation, a fusion, a truncation, an amplification, or a combination thereof).

For example, in certain embodiments, the biological sample preparation method includes capturing fragments of cell free DNA (cfDNA) with capture probes, converting the captured DNA fragments into circular DNA, and amplifying the circular DNA by performing rolling circle amplification (RCA). In particular, it is presently found that by performing this sample preparation method, it is possible to more successfully distinguish true alterations (e.g., aberrant methylation status and/or genomic mutations) from technical/sequencing artifacts. Moreover, it is presently found that samples prepared via this sample preparation method are more amenable to use of third generation sequencing to sequence the cfDNA. Third generation sequencing (also known as long-read sequencing) produce substantially longer reads than next generation sequencing (NGS). In certain embodiments, reads are at least 900 bases, at least 1 kb, at least 2 kb, at least 10 kb, at least 20 kb, at least 50 kb, at least 100 kb, at least 200 kb, at least 500 kb, at least 900 kb, at least 1Mb or more. In certain embodiments, the sequencing technology is single molecule real time sequencing (SMRT) (e.g., from Pacific Biosciences), nanopore technology (e.g., from Oxford), and Tru-seq Synthetic Long-Read technology (e.g., from Illumina).

An example third generation sequencing technology is nanopore DNA sequencing (e.g., Oxford Nanopore Technologies' systems, Oxford Science Park, UK), which provides significantly longer read lengths (e.g., well over 1 kb, up to 900 kb) compared to NGS systems (e.g., max read length from 150 to 300 bp). In certain embodiments described herein, the preparation methods described are particularly suitable for nanopore DNA sequencing.

In certain embodiments, the cfDNA is extracted from the biological sample (e.g., plasma, blood, serum, urine, stool, or tissue) and converted prior to DNA fragment capture. In certain embodiments, the capture probes are methylation capture probes and/or mutation capture probes, wherein the capture probes target one or more genomic regions (e.g., differentially methylated regions, DMRs) in a genome of interest. In certain embodiments, the captured DNA fragments are converted into circular double stranded DNA (dsDNA) and/or circular single stranded DNA (ssDNA) via DNA circularization (e.g., wherein the circular ssDNA is complementary to the original cfDNA strand). In certain embodiments, the circular DNA is amplified by performing rolling circle amplification (RCA). In certain embodiments, the method further includes sequencing the cfDNA using the amplified circular DNA, for example, using a third generation/next generation sequencing technique. In certain embodiments, the method further includes performing methylation target evaluation, mutation target evaluation, or simultaneous methylation and mutation target evaluation from the sequencing results.

In various embodiments, the present disclosure provides methods for detecting cancer (e.g., colorectal cancer and/or advanced adenoma) that include analysis of one or more methylation biomarkers in cell-free DNA (e.g., circulating tumor DNA, ctDNA) of a subject. In various embodiments, the present disclosure provides methods for cancer detection (e.g., colorectal cancer detection and/or advanced adenoma detection) that includes determining the methylation status of one or more methylation biomarkers in DNA e.g., cfDNA, for example, using a next generation sequencing (NGS) technique and/or a third generation sequencing technique (e.g., a targeted sequencing technique, a hybrid-capture based technique). Various methods provided herein are useful in cancer screening by analysis of an accessible biological sample of a subject, e.g., plasma, blood, serum, urine, stool, or tissue. In certain embodiments, cell-free DNA is obtained from a sample containing a tissue sample that is blood or a blood component (e.g., cfDNA, e.g., ctDNA).

In various embodiments, the methods described herein include screening for mutations of one or more mutation markers in cfDNA e.g., ctDNA. Mutations identified through detection methods described herein may be used to further classify and/or diagnose a disease or condition in combination with the methylation status(es) of the methylation biomarkers. For example, the presence of mutations in mutation markers and methylation status(es) of methylation markers may be acquired (e.g., simultaneously) in the same assay (e.g., a NGS assay or a third generation sequencing assay) conducted on a single sample. Obtaining information corresponding to methylation and mutation markers in the same assay allows for decreased costs and increased efficiency by not having to conduct separate assays. Additionally or alternatively, mutation markers may allow for further classification of a disease or condition (e.g., cancer). The presence and/or absence of one or more mutations may also allow for identification or recommendation of therapies for treatment of the disease and/or condition.

In various embodiments, the present disclosure relates to methods and/or systems for identifying methylation status of a methylation biomarker in cfDNA of a subject (e.g., a human subject) and/or detecting (e.g., screening for) a disease and/or condition (e.g., cancer) based on the methylation status of one or more known biomarkers. In certain embodiments, read-wise methylation values obtained from reads of methylation biomarkers are used to identify or diagnose a disease, e.g., using a classification model. In certain embodiments, a read-wise methylation value for a methylation biomarker may be based on a comparison of a number of methylated reads of a control DNA sample not affected by the disease and/or condition (e.g., cfDNA from a “healthy” subject, buffy coat DNA, DNA from a “healthy” tissue) as compared to a number of methylated reads of a pathological DNA sample affected by the disease or condition (e.g., cfDNA, e.g., ctDNA).

In certain embodiments, read-wise methylation values are based at least on a ratio of a total number of methylated CpG sites and a total number of CpG sites for each read corresponding to the methylation locus, wherein a read is a sequenced segment of a DNA fragment corresponding to the methylation locus.

In various embodiments, the present disclosure relates to methods and/or systems to obtain read-wise methylation values of one or more target biomarkers (e.g., DMRs) using third generation sequencing data and/or next generation (NGS) sequencing data. While it is understood that methylation status of individual markers may change in DNA of a subject afflicted by a disease, current bioinformatics-based tools used to identify abnormal methylation are insufficient to accurately detect abnormal methylation patterns. For example, current tools are not sufficiently sensitive to changes in methylation states between control and disease states to detect significant methylation changes in methylation markers. Additionally, such tools suffer from high signal to noise ratio, particularly when using cfDNA as a sample source as, in certain diseases, the amount of cfDNA in a sample may be small in blood or plasma samples. A read-wise assessment of methylation allows for a more appropriate identification and assessment of methylation. Exemplary assessment techniques for identifying and evaluating methylation and mutations are described in, for example, U.S. Provisional Patent Application No. 63/189,001 filed on May 14, 2021 and U.S. patent application Ser. No. 17/744231 filed on May 13, 2022, the disclosures of which is incorporated by reference in their entireties.

In various embodiments, the present disclosure relates to methods and/or systems for conducting third generation sequencing and/or next-generation sequencing (NGS) on samples of DNA, e.g., cfDNA. NGS sequencing on DNA samples is typically conducted using standard sets of manufactured kits and techniques. However, standard NGS techniques may insufficiently cover target regions, particularly as GC content of regions may vary widely from region to region. For example, methylation markers may have high GC content while mutation markers may have low GC content. Under certain NGS sequencing conditions, variations in GC content may lead to over-representation of regions having high GC content and/or underrepresentation of low GC content regions. Steps taken to improve GC coverage of high GC content regions may, in turn, lower coverage of low GC content regions (or vice versa). In addition, current NGS sequencing techniques lack sufficient means for determining data quality of samples.

It is found that these sources of error due to current NGS sequencing techniques may be eliminated or diminished by using the sample preparation method described herein and sequencing the cfDNA via third generation sequencing. The sample preparation method described, a specific example of which is presented herein, is found to be more amenable to the use of third generation sequencing to sequence the cfDNA than prior sample preparation methods. Specific capture probes used and their ratios may be designed, for example, to enrich for either only methylated reads or for unmethylated reads in a certain target region, thereby reducing (or eliminating) non-informative reads and enhancing the cancer-distinguishing signal against background noise.

### Illustrative Embodiment of Sample Preparation

### EXAMPLE 1

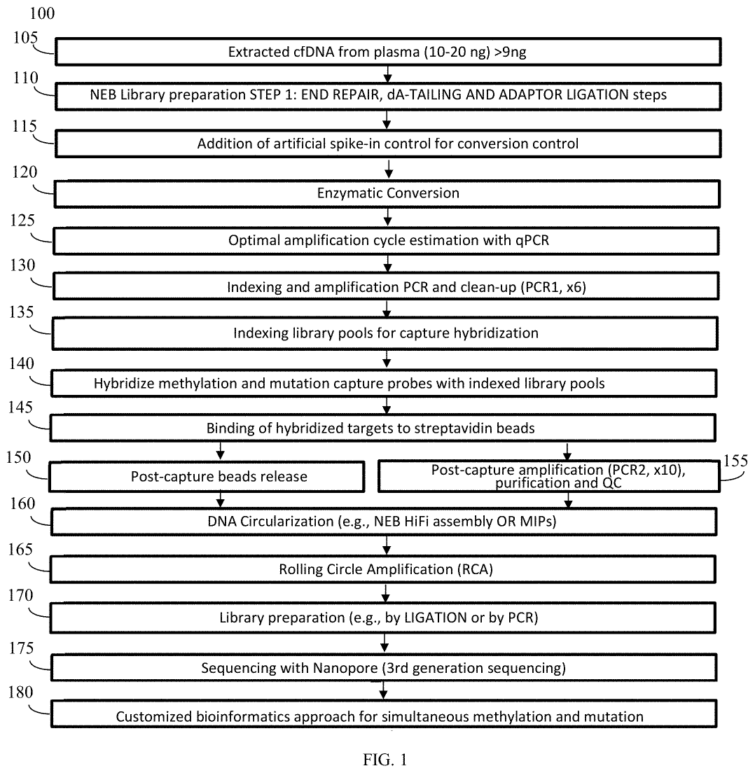

Described herein is an illustrative embodiment of preparation of a sample for DNA sequencing. An illustrative overview of the process is provided in, for example, FIG. 1. FIG. 1 is a general workflow (100) of a hybrid capture based targeted methylation nanopore sequencing approach, according to an illustrative embodiment.

DNA (e.g., cfDNA, ctDNA) is extracted from a plasma sample (e.g., human plasma) (105). In certain embodiments, at least 9 ng of plasma is used in the methods described herein. In certain embodiments from about 10 ng to about 20 ng of DNA is extracted from plasma. In certain embodiments, the volume of plasma sample acquired is at least 1mL (e.g., at least 2mL, at least 3mL, at least 4mL, at least 5mL or more).

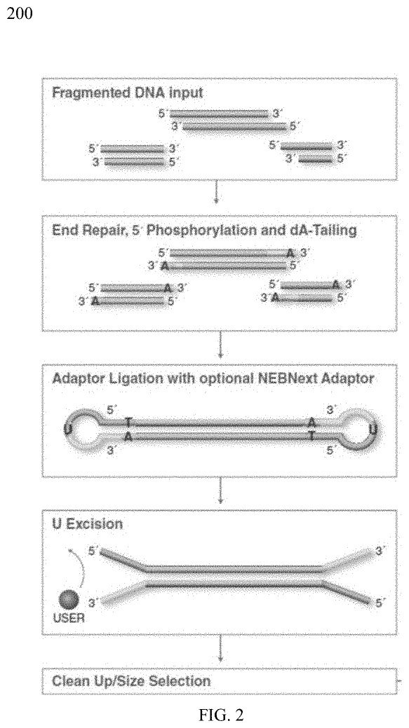

The extracted DNA undergoes a library preparation process (110) (e.g., a first part of the library preparation process). In certain embodiments, the library preparation process involves end repair (e.g., 5′ Phosphorylation and dA-tailing) and adaptor ligation. In certain embodiments, library preparation involves the workflow (200) depicted in FIG. 2. Fragments of DNA from prior steps in the method are used as input. In certain embodiments, a NEBNext® Ultra™ II DNA Library Prep Kit for Illumina is used.

In certain embodiments, an artificial spike-in control (115) for conversion control (e.g., as described herein) is added. In certain embodiments, the artificial spike-in control is added prior to conversion. In certain embodiments, artificially methylated and unmethylated spike-in (e.g., Premium RRBS kit [Diagenode]) control sequences are added to cfDNA samples prior to conversion of the cfDNA. In certain embodiments, the spike-in control sequences are added in using a 1:10000 ratio (e.g., by volume) of spike-in control to cfDNA.

DNA is subjected to enzymatic conversion (120) to deaminate cfDNA. Deamination of the cfDNA helps in identification of methylated and unmethylated cytosine residues, particularly at CpG sites. In certain embodiments, the enzymatic conversion method used is from the NEB enzymatic conversion kit NEB E7120.

The optimal number of amplification cycles is then estimated with qPCR (125). In certain embodiments, optimal library amplification is assessed by qPCR using KAPA SYBR® FAST (Sigma-Aldrich) on LightCycler® 96 System (Roche). qPCR can be used to measure a total concentration of a prepared library (e.g., as described herein). qPCR determines the optimal number of PCR cycles that may need to be performed in order to obtain the minimum amount of library material. In certain embodiments, generated libraries are assessed with RNA 6000 Pico Kit on a Fragment Analyzer™ (Agilent).

Then, indexing library pools are created for capture hybridization (135). The method involves hybridizing methylation and/or mutation capture probes with indexed library pools (140).

Hybridized targets are bound to streptavidin beads (145). In certain embodiments, the targets are released from the beads (150) (e.g., without PCR amplification). In certain embodiments, targets are released from beads using basic conditions. In certain embodiments, the targets are amplified with PCR post-capture (155). In certain embodiments, at least 5, at least 6, at least 7, at least 8, at least 9, at least 10 or more PCR cycles are used to amplify the DNA. In certain embodiments, the PCR amplified targets are then purified and qc (quality control) steps are performed.

The sample DNA is then circularized (160) prior to performing rolling circle amplification (RCA) (165). In certain embodiments, the fragments of DNA prior to the addition of DNA splints and circularization are from about 150 to 1000 bp (e.g., from about 300 to about 500 bp, from about 375 to about 425 bp). In certain embodiments, a sample of DNA is at least 2 ng/μl (e.g., at least 3 ng/μl) if PCR is used to amplify the captured DNA (e.g., using 10× PCR cycles). In certain embodiments, DNA is circularized using a HiFi NEB assembly kit (e.g., NEBuilder® HiFi DNA Assembly kit). In certain embodiments, an about 1:2 molar ratio of sample DNA (e.g., hybrid captured DNA, PCR amplified DNA) to splint DNA is used (e.g., about 1:2, about 1:3, about 1:4, about 1:5 molar ratio) (e.g., from 1:1 to 1:6 molar ratio, from 1:2 to 1:5 molar ratio). In certain embodiments, DNA is circularized using MIPs (Molecular Inversion Probes).

After circularization, the circularized DNA [e.g., circularized single stranded DNA (ssDNA), circularized double stranded DNA (dsDNA)] is amplified using rolling circle amplification (RCA) (165) (e.g., as described herein). RCA is a method of amplification of circular DNA molecules.

A library preparation step (170) follows RCA. In certain embodiments, library preparation is performed using ligation (e.g., end-repair and adapter ligation). In certain embodiments, library preparation is performed using PCR (e.g., end-repair, PCR adapter ligation, and PCR).

In certain embodiments, the method involves sequencing using a 3^{rd} generation sequencing technique. In certain embodiments, the sequencing technique is Nanopore sequencing.

In certain embodiments, customized bioinformatics approaches are used to evaluate the sequencing results (180). Bioinformatics approaches used to evaluate methylation and/or mutation markers described in U.S. Provisional Patent Application No. 63/189,001 filed on May 14, 2021, U.S. patent application Ser. No. 17/744231 filed on May 13, 2022, or U.S. application Ser. No. 17/027,148 filed Sep. 21, 2020, the disclosures of which are incorporated by reference in their entireties.

### EXAMPLE 2

cfDNA Extraction from Plasma and Quality Control Samples (e.g., FIG. 1, Step 105)

An illustrative embodiment of cfDNA extraction from plasma is described below.

4-5 ml of human plasma is used for cfDNA is extraction. In certain embodiments, a manual protocol follows the manufacturer's specifications for QlAamp® MinElute® ccfDNA Mini Kit as described herein.

In detail:

1. Mix components in a 15 ml tube; incubate 10 min at RT (room temperature) (15—Plasma (ml) Magnetic Bead Suspension (μl) Proteinase K (μl) Bead Binding Buffer (μl))

TABLE 1
Components for cfDNA extraction from plasma.

| | Magnetic Bead | | |
|---|---|---|---|
| Plasma | Suspension | Proteinase K | Bead Binding Buffer |
| (ml) | (μl) | (μl) | (μl) |
| 4 | 120 | 220 | 600 |
| 5 | 150 | 275 | 650 |

2. Spin briefly (30 s at 200×g) to remove any solution in cap. Place tube with bead solution on a 15 ml magnetic rack. Incubate at least 1 min, until solution is clear. Discard supernatant.

3. Remove tube from 15 ml magnetic rack. Add 200 μl Bead Elution Buffer to bead pellet; vortex. Pipet up and down to mix and rinse tube wall. Transfer bead mixture into Bead Elution Tube. Incubate 5 min on thermomixer at RT, 300 rpm.

4. Place Bead Elution Tube with bead solution on a 2 ml magnetic rack. Incubate at least 1 min, until solution is clear.

5. Transfer supernatant into new Bead Elution Tube and discard bead pellet.

6. Add 300 μl Buffer ACB to supernatant; vortex to mix. Briefly centrifuge tube.

7. Apply mixture from step 6 to QIAamp® UCP MinElute® Column and centrifuge 1 min at 6000×g. Place QIAamp® UCP MinElute Column in a clean 2 ml collection tube, and discard flow-through.

8. Add 500 μl buffer ACW2 to QIAamp® UCP MinElute® Column; centrifuge 1 min at 6000×g. Place QIAamp® UCP MinElute® Column in a clean 2 ml collection tube; discard flow-through. Centrifuge 3 min at full speed (20,000×g; 14,000 rpm).

9. Place QIAamp® UCP MinElute® Column in a clean 1.5 ml elution tube; discard 2 ml collection tube from step 8. Open lid; incubate 3 min at 56° C.

10. Apply 20-80 μl Ultraclean water to membrane center. Close lid; incubate 1 min at RT (room temperature).

11. Centrifuge 1 min at full speed (20,000×g; 14,000 rpm).

12. Place the QIAamp® UCP MinElute® Column in a clean 1.5 ml elution tube. Aspirate the eluate in the 1.5 ml elution tube from step 9 and reload it onto the center of the membrane. Close the lid, and incubate 1 min at RT. Centrifuge 1 min at full speed (20,000×g; 14,000 rpm).

### NEB Library Preparation STEP 1 (e.g., FIG. 1, Step 110)

Between 10 ng and 20 ng of cfDNA is used for library preparation using the NEBNext® Ultra™ II DNA Library Prep Kit for Illumina.

FIG. 2 is an illustrative method 200 for end repair, dA-tailing and adaptor ligation used herein.

13. Add the following components to a sterile nuclease- free tube as shown in Table 2 below:

TABLE 2
Components for Library Preparation

| COMPONENT | VOLUME |
|---|---|
| o (green) NEBNext ® Ultra ™ II End Prep Enzyme Mix | 3 μl |
| o (green) NEBNext ® Ultra ™ II End Prep Enzyme Mix | 7 μl |
| Fragmented DNA | 50 μl |
| Total Volume | 60 μl |

Place in a thermocycler, with the heated lid set to ≥75° C.

30 minutes@20° C.

30 minutes@65° C.

Hold at 4° C.

14. Dilute Adaptor as follows in Table 3 below:

TABLE 3
Adaptor dilution components.

| | ADAPTOR DILUTION | WORKING |
|---|---|---|
| | (VOLUME OF ADAPT- | ADAPTOR |
| INPUT | OR:TOTAL VOLUME) | CONCENTRATION |
| 1 μg-101 ng | No Dilution | 15 μM |
| 100 ng-5 ng | 10-Fold (1:10) | 1.5 μM |
| less then 5 ng | 25-Fold (1:25) | 0.6 μM |

15. Add the following components from Table 4 directly to the End Prep Reaction Mixture, using EM-seq Adaptor:

TABLE 4
Components added to End Prep Reaction Mixture.

| COMPONENT | VOLUME | |
|---|---|---|
| End Prep Reaction Mixture (Step 1.3 in Section 1) | 60 | μl |
| (red) NEBNext ® Adaptor for Illumina** | 2.5 | μl |
| (red) NEBNext ® Ultra II Ligation Master Mix* | 30 | μl |
| (red) NEBNext ® Ligation Enhancer | 1 | μl |
| Total Volume | 93.5 | μl |

16. Incubate at 20° C. for 15 minutes in a thermocycler with the heated lid off.

17. Add 3μl of (red) USER® Enzyme to the ligation mixture.

18. Mix well and incubate at 37° C. for 15 minutes with the heated lid set to ≥47° C.

19. Purify DNA with beads in 28 μl elution buffer (no size selection for DNA input <50 ng).

### Neb Enzymatic Conversion (NEB E7120) (e.g., FIG. 1, Step 120)

Add artificially methylated and unmethylated spike-in to all cfDNA prior to conversion (using a 10K-ratio) to evaluate the conversion efficiency (e.g., FIG. 1, step 115).

Oxidation of 5-Methylcytosines and 5-Hydroxymethylcytosines

20. On ice, add the following components listed in Table 5 directly to the 28 μl EM-seq adaptor ligated DNA from step 19.

TABLE 5
Components to add to EM-seq adaptor ligated DNA from Step 19.

| COMPONENT | VOLUME |
|---|---|
| o (yellow) TET2 Reaction Buffer (TET2 Reaction Buffer | 10 μl |
| plus reconstituted TET2 Reaction Buffer Supplement) | |
| o (yellow) Oxidation Supplement | 1 μl |
| o (yellow) DTT | 1 μl |
| o (yellow) Oxidation Enhancer | 1 μl |
| o (yellow) TET2 | 4 μl |

21. Dilute the 500 mM Fe(II) Solution (yellow) by adding 1 μl of the solution to 1249 μl of water.

22. Combine Diluted Fe(II) Solution and EM-seq DNA as shown in Table 6 below with Oxidation Enzymes.

TABLE 6
Combination of Diluted Fe(II) Solution and EM-seq DNA.

| COMPONENT | VOLUME |
|---|---|
| EM-seq DNA (from step 20) | 45 μl |
| Diluted Fe(II) Solution (from step 21) | 5 μl |
| Total Volume | 50 μl |

23. Incubate at 37° C. for 1 hour in a thermocycler with the heated lid set to ≥45° C.

24. Transfer the samples to ice and add 1μl of Stop Reagent (yellow).

25. Incubate at 37° C. for 30 minutes then at 4° C. in a thermocycler with the heated lid set to ≥45° C.

Denaturation of DNA

26. Vortex Sample Purification Beads to resuspend. SPRIselect or AMPure XP Beads can be used as well. If using AMPure XP Beads, allow the beads to warm to room temperature for at least 30 minutes before use.

27. Add 90 μl of resuspended NEBNext® Sample Purification Beads to each sample. Mix well by pipetting up and down at least 10 times. Be careful to expel all of the liquid out of the tip during the last mix.

28. Incubate samples on bench top for at least 5 minutes at room temperature.

29. Place the tubes against an appropriate magnetic stand to separate the beads from the supernatant.

30. After 5 minutes (or when the solution is clear), carefully remove and discard the supernatant. Be careful not to disturb the beads that contain DNA targets (Caution: do not discard the beads).

31. Add 200 μl of 80% freshly prepared ethanol to the tubes while in the magnetic stand. Incubate at room temperature for 30 seconds, and then carefully remove and discard the supernatant. Be careful not to disturb the beads that contain DNA targets.

32. Repeat the wash once for a total of two washes. Be sure to remove all visible liquid after the second wash using a p10 pipette tip.

33. Air dry the beads for up to 2 minutes while the tubes are on the magnetic stand with the lid open.

34. Caution: Do not over-dry the beads. This may result in lower recovery of DNA target. Elute the samples when the beads are still dark brown and glossy looking, but when all visible liquid has evaporated. When the beads turn lighter brown and start to crack they are too dry.

35. Remove the tubes from the magnetic stand. Elute the DNA target from the beads by adding 17 μl of Elution Buffer (white).

36. Mix well by pipetting up and down 10 times. Incubate for at least 1 minute at room temperature. If necessary, quickly spin the sample to collect the liquid from the sides of the tube before placing back on the magnetic stand.

37. Place the tube on the magnetic stand. After 3 minutes (or whenever the solution is clear), transfer 16 μl of the supernatant to a new PCR tube.

38. Safe Stopping Point: Samples can be stored overnight at −20° C.

39. Pre-heat thermocycler to 85° C.

40. Add 4 μl Formamide to the 16 μl of oxidized DNA. Vortex to mix or by pipetting up and down at least 10 times, centrifuge briefly.

41. Incubate at 85° C. for 10 minutes in the pre-heated thermocycler with the heated lid on.

42. Immediately place on ice.

Deamination of Cytosines

43. On ice, add the following components to the 20 μl of denatured DNA as shown in Table 7.

TABLE 7
Deamination components.

| COMPONENT | VOLUME |
|---|---|
| Nuclease-free water | 68 μl |
| o (orange) APOBEC Reaction Buffer | 10 μl |
| o (orange) BSA | 1 μl |
| o (orange) APOBEC | 1 μl |
| Total volume | 100 μl |

44. Mix thoroughly by vortexing or by pipetting up and down at least 10 times, centrifuge briefly.

45. Incubate at 37° C. for 3 hours then at 4° C. in a thermocycler with the heated lid set to ≥45° C. or on.

46. Safe Stopping Point: Samples can be stored overnight at either 4° C. in the thermocycler or at −20° C. in the freezer.

47. Caution: The Sample Purification Beads behave differently during the APOBEC clean-up. After the bead washes, do not overdry the beads as they become very difficult to resuspend.

48. Vortex Sample Purification Beads to resuspend. SPRIselect or AMPure XP Beads can be used as well. If using AMPure XP Beads, allow the beads to warm to room temperature for at least 30 minutes before use.

49. Add 100 μl of resuspended NEBNext® Sample Purification Beads to each sample. Mix well by pipetting up and down at least 10 times. Be careful to expel all of the liquid out of the tip during the last mix.

50. Incubate samples on bench top for at least 5 minutes at room temperature.

51. Place the tubes against an appropriate magnetic stand to separate the beads from the supernatant.

52. After 5 minutes (or when the solution is clear), carefully remove and discard the supernatant. Be careful not to disturb the beads that contain DNA targets (Caution: do not discard the beads).

53. Add 200 μl of 80% freshly prepared ethanol to the tubes while in the magnetic stand. Incubate at room temperature for 30 seconds, and then carefully remove and discard the supernatant. Be careful not to disturb the beads that contain DNA targets.

54. Repeat the wash once for a total of two washes. Be sure to remove all visible liquid after the second wash using a p10 pipette tip.

55. Air dry the beads for up to 90 seconds while the tubes are on the magnetic stand with the lid open.

56. Caution: Do not over-dry the beads. This may result in lower recovery of DNA target. Elute the samples when the beads are still dark brown and glossy looking, but when all visible liquid has evaporated. When the beads turn lighter brown and start to crack they are too dry.

57. Remove the tubes from the magnetic stand. Elute the DNA target from the beads by adding 21 μl of Elution Buffer (white).

58. Mix well by pipetting up and down 10 times. Incubate for at least 1 minute at room temperature. If necessary, quickly spin the sample to collect the liquid from the sides of the tube before placing back on the magnetic stand.

59. Place the tube on the magnetic stand. After 3 minutes (or whenever the solution is clear), transfer 20 μl of the supernatant to a new PCR tube.

60. Safe Stopping Point: Samples can be stored overnight at −20° C.

61. Converted cfDNA quality was assessed using RNA 6000 Pico Kit (Agilent) on a Fragment AnalyzerTM (Agilent).

### Neb Library Prep Step2 (PCR1) (e.g., FIG. 1, Step 130)

62. Add the following shown in Table 8 on ice to 20 μl converted DNA from step 59.

TABLE 8
NEB Library Preparation Components.

| COMPONENT | VOLUME |
|---|---|
| EM-seq Index Primer*^{,}** | 5 μl |
| o (blue) NEBNext ® Q5U Master Mix | 25 μl |
| Total volume | 50 μl |

Thermocycler settings for the mixture are presented below in Table 9.

TABLE 9
Thermocycler settings.

| CYCLE STEP | TEMP | TIME | CYCLES |
|---|---|---|---|
| Initial Denaturation | 98° C. | 30 seconds | 1 |
| Denaturation | 98° C. | 10 seconds | 6 |
| Annealing | 62° C. | 30 seconds | |
| Extension | 65° C. | 60 seconds | |
| Final Extension | 65° C. | 5 minutes | 1 |
| Hold | 4° C. | ∞ | |

63. Purify DNA.

64. Multiplex 8 samples together, 187.5 ng each (total 1.5ᵢig DNA). In certain embodiments, the amount of purified DNA is increased to 250 ng/sample.

### Hybrid Capture

Thaw all required reagents on ice, then pulse-vortex for 2 seconds to mix and pulse- spin.

In preparation for Hybridize Capture Probes with Pools, also thaw on ice: From the Twist Fast Hybridization Reagents:

Fast Hybridization Mix

Hybridization Enhancer

65. Transfer the calculated volumes from each amplified indexed library to a hybridization reaction tube (either a 0.2-ml thin-walled PCR strip-tube or 96- well plate) for each hybridization reaction to be performed.

Prepare the Pre-Hybridization Solution

66. Add the following volumes of reagents shown in Table 10 to each amplified indexed library to create a pre-hybridization solution. Mix by flicking the tube(s).

TABLE 10
Pre-hybridization solution reagents.

| REAGENT | VOLUME |
|---|---|
| Twist Probe Panel | 4 μl |
| Optional: Secondary Panel (if a secondary panel is not used, | 4 μl |
| do not add water as the entire solution will be dried down) | |
| Universal Blockers | 8 μl |
| Blocker Solution | 5 μl |

67. Pulse-spin the tube(s) and ensure there are minimal bubbles present.

68. Dry the pre-hybridization solution (library, probes, blockers) in the tube(s) used for the hybridization reaction using a SpeedVac system (or a similar evaporator device) using low or no-heat.

69. Program a 96-well thermal cycler with the following conditions in Table 11 and set the heated lid to 85° C.:

TABLE 11
Pre-hybridization conditions.

| Steps | Temperature | Time |
|---|---|---|
| Step 1 | 95° C. | HOLD |
| Step 2 | 95° C. | 5 minutes |
| Step 3 | 60° C. | 15 minutes to 4 hours¹ |

Resuspend the Pre-Hybridization Solution

70. Heat the Fast Hybridization Mix at 65° C. for 10 minutes, or until all precipitate is dissolved. Vortex and use immediately. Do not allow the Fast Hybridization Mix to cool to room temperature.

71. Resuspend the dried pre-hybridization solution from Step 4 in 20 μl Fast Hybridization Mix.

72. Pulse-spin the tube(s) and ensure there are no bubbles present.

73. Add 30 μl Hybridization Enhancer to the top of the pre-hybridization solution.

74. Pulse-spin the tube(s) to ensure all solution is at the bottom of the tube(s).

75. Transfer the tube(s) to the preheated thermal cycler and move to Steps 2 and 3 of the thermocycler program.

### Bind Hybridized Targets to Streptavidin Beads (e.g., FIG. 1, Step 145)

Prepare the Beads

76. Vortex the pre-equilibrated Streptavidin Binding Beads until mixed.

77. Add 100 μl Streptavidin Binding Beads to a 1.5-ml microcentrifuge tube. Prepare one tube for each hybridization reaction.

78. Add 200 μl Fast Binding Buffer to the tube(s) and mix by pipetting.

79. Place the tube(s) on a magnetic stand for 1 minute, then remove and discard the clear supernatant. Make sure to not disturb the bead pellet. Remove the tube from the magnetic stand.

80. Repeat the wash (Steps 78 and 79) two more times for a total of three washes.

81. After removing the clear supernatant from the third wash, add a final 200 μl Fast Binding Buffer and resuspend the beads by vortexing until homogenized.

82. After the hybridization is complete (Step 75), open the thermal cycler lid and quickly transfer the volume of each hybridization reaction including Hybridization Enhancer into a corresponding tube of washed Streptavidin Binding Beads from Step 81. Mix by pipetting and flicking.

NOTE: Rapid transfer directly from the thermal cycler at 60° C. is a critical step for minimizing off-target binding. Do not remove the tube(s) of hybridization reaction from the thermal cycler or otherwise allow it to cool to less than 60° C. before transferring the solution to the washed Streptavidin Binding Beads.

Bind the Targets

83. Mix the tube(s) of the hybridization reaction with the Streptavidin Binding Beads for 30 minutes at room temperature on a shaker, rocker, or rotator at a speed sufficient to keep the solution mixed.

NOTE: Do not vortex. Aggressive mixing is not required.

84. Remove the tube(s) containing the hybridization reaction with Streptavidin Binding Beads from the mixer and pulse-spin to ensure all solution is at the bottom of the tube(s).

85. Place the tube(s) on a magnetic stand for 1 minute.

86. Remove and discard the clear supernatant including the Hybridization Enhancer. Do not disturb the bead pellet.

NOTE: A trace amount of Hybridization Enhancer may be visible after supernatant removal and throughout each wash step. It will not affect the final capture product.

87. Remove the tube(s) from the magnetic stand and add 200 pl preheated Fast Wash Buffer 1. Mix by pipetting.

88. Incubate the tube(s) for 5 minutes at 70° C.

89. Place the tube(s) on a magnetic stand for 1 minute.

90. Remove and discard the clear supernatant. Make sure to not disturb bead pellet.

91. Remove the tube(s) from the magnetic stand and add an additional 200 pl of preheated Fast Wash Buffer 1. Mix by pipetting.

92. Incubate the tube(s) for 5 minutes at 70° C.

NOTE: The temperature of this 70° C. Wash Buffer 1 can be altered to tune off-target and uniformity in a use-case specific manner.

93. Pulse-spin to ensure all solution is at the bottom of the tube(s).

94. Transfer the entire volume from Step 93 (-200 pl) into a new 1.5-ml microcentrifuge tube, one per hybridization reaction. Place the tube(s) on a magnetic stand for 1 minute.

NOTE: A tube transfer is required at this step as it reduces background due to non-targeted library that may stick to the surface of the tube.

95. Remove and discard the clear supernatant. Make sure to not disturb the bead pellet.

96. Remove the tube(s) from the magnetic stand and add 200 μl of 48° C. Wash Buffer 2. Mix by pipetting, then pulse-spin to ensure all solution is at the bottom of the tube(s).

97. Incubate the tube(s) for 5 minutes at 48° C.

98. Place the tube(s) on a magnetic stand for 1 minute.

99. Remove and discard the clear supernatant. Make sure to not disturb the bead pellet.

100. Repeat the wash (Steps 96-99) two more times, for a total of three washes.

101. After the final wash, use a 10 μl pipette to remove all traces of supernatant. Proceed immediately to the next step. Do not allow the beads to dry.
OPTION 1: Basic conditions beads detachment (without PCR amplification) (e.g., FIG. 1, step 150)

102. After the final wash, use a 10 μL pipette to remove all traces of supernatant. Proceed immediately to the next step. Do not allow the beads to dry.

NOTE: Before removing supernatant, the bead pellet may be briefly spun to collect supernatant at the bottom of the tube or plate and returned to the magnetic plate.

103. Remove the tube(s) from the magnetic stand and add 40 μL of freshly prepared 100 mM NaOH (4E-6 moles base; 4.0 μmol total) to washed beads.

104. Incubate at room temperature with agitation for 02:30 (mm:ss) (minutes:seconds).

105. Briefly spin down and put on a magnet. Remove supernatant and place on ice.

106. Immediately add 4.2 μL of freshly prepared 1 M glacial acetic acid (4.2E-6 moles acid; 4.2 μmol total) and 0.8 μL water (final volume of 45 μL).

NOTE: The acetic acid can also be premixed into water (42 μL of 1M acetic acid +8 uL of water to create a 840mM working stock). 5 μL of this solution can be added directly to the 40 μL NaOH elution. 5 mL of 1M glacial acetic acid can be prepared in the following manner; slowly add 0.287 mL of neat acetic acid to 1.25 mL deionized water. Adjust the final volume of solution to 5 mL with deionized water.
OPTION 2: Post-capture PCR amplify, purify, and perform qc (only if 90 ng DNA are required later for DNA circularization) (e.g., FIG. 1, step 155)

102. Remove the tube(s) from the magnetic stand and add 45 μl water. Mix by pipetting until homogenized, then incubate this solution, hereafter referred to as the Streptavidin Binding Bead slurry, on ice.

103. Transfer 22.5 μl of the Streptavidin Binding Bead slurry to a 0.2-ml thin-walled PCR strip-tube(s). Keep on ice until ready to use in the next step.

NOTE: Store the remaining 22.5 μl water/Streptavidin Binding Bead slurry at—20° C. for future use.

104. Prepare a PCR mixture by adding the following reagents in Table 12 to the tube(s) containing the Streptavidin

TABLE 12
Reagents for PCR mixture.

| Reagent | Volume Per Reaction |
|---|---|
| Strepavidin Binding Bead Slurry | 22.5 μl |
| Amplification Primers ILMN | 2.5 μl |
| KAPA HiFi HotStart ReadyMix | 25 μl |
| Total | 50 μl |

PCR conditions (10x cycles or less, to be determined based on minimum amount of DNA required for circularization). Table 13 below shows the PCR steps of a thermocycler with Table 14 showing variations in the thermocycler program based on the panel size.

TABLE 13
Thermocycler Steps.

| STEP | TEMPERATURE | TIME | Number of cycles |
|---|---|---|---|
| 1. Initialization | 98° C. | 45 seconds | 1 |
| 2. Denaturation | 98° C. | 15 seconds | Varies |
| Annealing | 60° C. | 30 seconds | |
| Extension | 72° C. | 30 seconds | |
| 3. Final Exension | 72° C. | 1 minute | 1 |
| 4. Final Hold | 4° C. | HOLD | — |

TABLE 14
Custom Panel Size Variations.

| | |
|---|---|
| Custom Panel Size | Number Cycles |

| | | |
|---|---|---|
| >100 | Mb | 5 |
| 50-100 | Mb | 7 |
| 10-50 | Mb | 8 |
| 1-10 | Mb | 9 |
| 500-1,000 | kb | 11 |
| 100-500 | kb | 13 |
| 5-100 | kb | 14 |
| <50 | kb | 15 |

105. When the thermocycler program is complete, remove the tube(s) from the block and immediately purify DNA.

106. Vortex the DNA Purification Beads to mix.

107. Add 90 μl (1.8X) homogenized DNA Purification Beads to the tube(s) from Step 46. Mix well by vortexing.

NOTE: It is not necessary to recover supernatant or remove Streptavidin Binding Beads from the amplified PCR product.

108. Incubate for 5 minutes at room temperature.

109. Place the tube(s) on a magnetic plate for 1 minute.

110. Without removing the tube(s) from the magnetic plate, remove and discard the clear supernatant.

111. Wash the DNA Purification Bead pellet with 200 μl freshly prepared 80% ethanol for 1 minute, then remove and discard the ethanol. Repeat this wash once, for a total of two washes, while keeping the tube on the magnetic plate.

112. Using a 10 μl pipet, remove all residual ethanol, making sure to not disturb the bead pellet.

113. Air-dry the bead pellet on a magnetic plate for 5-10 minutes or until the bead pellet is dry. Do not overdry the bead pellet.

114. Remove the tube(s) from the magnetic plate and add 32 μl water. Mix by pipetting until homogenized and incubate at room temperature for 2 minutes.

115. Place the tube(s) on a magnetic plate and let stand for 3 minutes or until the beads fully pellet.

116. Transfer 30 μl of the clear supernatant containing the enriched library to a clean thin-walled PCR 0.2-ml strip-tube, making sure not to disturb the bead pellet.

117. Validate and quantify each enriched library using an Agilent Bioanalyzer

118. High Sensitivity DNA Kit and a Thermo Fisher Scientific Qubit dsDNA High Sensitivity Quantitation Assay.

NOTE: When using the Agilent Bioanalyzer High Sensitivity DNA Kit, load 0.5 μl of the final sample.

### DNA Circularization (e.g., FIG. 1, Step 160)

119. Average fragment length should be 375-425 bp using a range setting of 150-1,000 bp. Final concentration should be ≥3 ng/μl in 300 if 10× PCR cycles are used. If PCR efficiency is optimal (i.e., 100%), 90 ng DNA would be produced after 10 PCR cycles if starting from 0.087 ng of DNA. 0.087 ng of DNA would be obtained after hybrid capture. If PCR is not performed, hybrid-captured DNA is still attached to Streptavidin-beads in 450 water. Concentration of the samples in volumes up to 100 will be required.

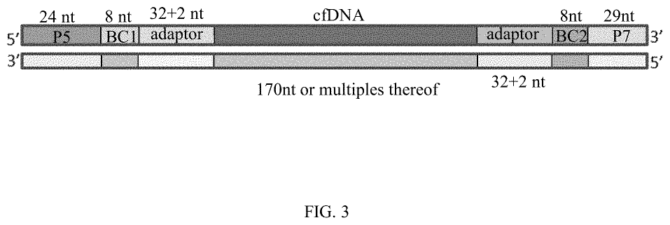

FIG. 3 shows an exemplary DNA segment to be circularized. P5 primer (24nt long) is shown joined to a barcode (BC1) segment, which is followed by an adaptor (32+2nt) and cfDNA fragment about 170nt long or multiples thereof. This is joined to a second adaptor segment (32+2nt), a second barcode segment (BC2; 8nt), and P7 primer (29nt).

### Option 1: DNA Circularization by HiFi NEB Assembly Kit

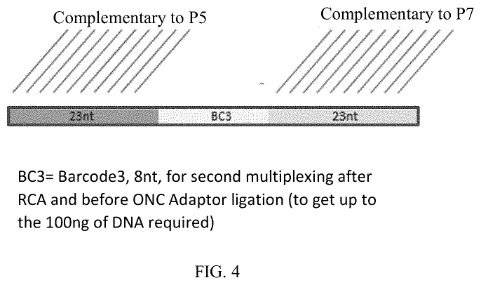

1. Design Splint DNA as follows (to be ordered as ssDNA) and shown in FIG. 4.

The Splint DNA has a first segment complementary to the P5 portion of DNA (23nt long), a segment of barcode DNA (BC3), and a second segment of DNA (23nt long) complementary to P7. The BC3 segment is for a second multiplexing after RCA and before ONC Adaptor ligation (to get up to the 10Ong of DNA required).

2. Mix 0.5 ng (0.0025pmol) (for hybrid-captured DNA without PCR step) or 90 ng (0.45pmol) DNA (for hybrid-captured DNA that has been PCR amplified) from Step 1 with splint DNA (1:2-1:5 molar ratio of, for example, hybrid-captured DNA : splint DNA) and bring to 100. Add 100 2x NEB HiFi assembly mix and incubate 60min at 55° C.

3. Digest noncircular DNA with lᵢ.il 1:10 Exonuclease III (10U, linear dsDNA specific) +1 μl Exonuclease I (20U, linear ssDNA specific) at 37° C. 30min (all from NEB).

4. Extract circularized DNA with NEB selection beads or SPRIN beads with cutoff to eliminate DNA<350bp.

5. Elute DNA in 31 μl water and measure circular dsDNA concentration.

RCA (Rolling Circle Amplification) (e.g., FIG. 1, step 165)

6. Prepare RCA reaction as follows in Table 15:

TABLE 15
RCA reaction solution.

| | |
|---|---|
| DNA solution from Step 5. | 30 μl |
| 10x Phi29 buffer | 5 μl |
| dNTPs each 10 mM | 1 μl |
| Primer Fw P5 10 μM | 2.5 μl |
| water | Up to 48.75 μl |

7. Heat reaction at 95° C. for 3 min, then immediately cool on ice 3-5min.

8. Add to the reaction the following as shown in Table 16:

TABLE 16
Reaction mixture additions.

| | |
|---|---|
| Phi29 pol (10 U/μl) NEB M0269 | 1 μl |
| BSA 20 mg/ml | 0.25 μl |

9. Incubate reaction at 30° C. for 30 min-2h-4h (to be tested for median product length).

10. After incubation, heat to 60° C. for 10 min. The resulting reaction mixture can be held at 4° C. prior to moving to Step 11.

Option 2: DNA Circularization by MIPs (e.g., FIG. 1, step 160)

2. Design Splint DNA as follow (to be ordered as ssDNA) (See FIG. 4).

Splint DNA has a first segment complementary to the P5 portion of DNA (23nt long), a segment of barcode DNA (BC3), and a second segment of DNA (23nt long) complementary to P7. The BC3 segment is for a second multiplexing after RCA and before ONC Adaptor ligation (to get up to the 10Ong of DNA required)

3. Mix on ice 0.5 ng (0.0025pmol) (for hybrid-captured DNA without PCR step) or 90 ng (0.45pmol) DNA (for hybrid-captured DNA PCR amplified) from Step 1 with splint DNA (1:1-1:5 molar ratio). Add 20 μl 2x Phusion Master Mix (NEB) +0.50 ampligase (10U/μl, Lucigen) +40 μl 10x ampligase buffer and bring up to 40 μl with water. Incubate reaction as follows in Table 17:

TABLE 17

| | | |
|---|---|---|
| 95° C. | 3 min | |
| 95° C. | 30 sec | 5x |
| X° C. (58-70° C.) | 1 min | |
| 37° C. | 2 min | |

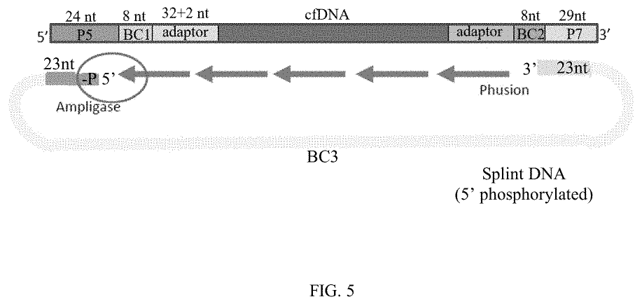

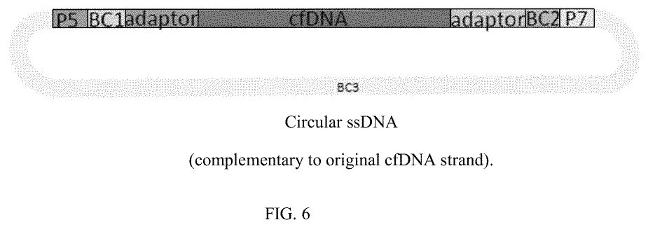

FIG. 5 shows integration of the DNA fragments of FIG. 3 and FIG. 4 together to form circularized DNA, which is shown in FIG. 6.

4. Digest noncircular DNA with 1 μl 1:10 Exonuclease III (10U, linear dsDNA specific) +10 Exonuclease I (20U, linear ssDNA specific) at 37° C. 30min (all from NEB).

5. Extract circularized DNA with NEB selection beads or SPRIN beads with cutoff to eliminate DNA<350bp.

6. Elute DNA in 310 water and measure circular ssDNA concentration.

RCA (Rolling Circle Amplification) (e.g., FIG. 1, step 165)

7. Prepare RCA reaction as follows in Table 18:

TABLE 18
RCA Amplification Solution.

| | | |
|---|---|---|
| DNA solution from Step 6. | 30 | μl |
| 10x Phi29 buffer | 5 | μl |
| dNTPs each 10 mM | 1 | μl |
| Primer against P5 10 μM | 2.5 | μl |
| Phi29 pol (10 U/μl) NEB M0269 | 1 | μl |
| BSA 20 mg/ml | 0.25 | μl |

| | |
|---|---|
| water | Up to 50 μl |

8. Incubate reaction at 30° C. 30min-2h-4h (to be tested for median product length, product will be ssDNA as the original cfDNA strand), then at 60° C. for 10 min. Samples can be held at 4° C. for an extended (i.e., indefinite) period of time. Go to step 11 below.

11. Purify DNA with cutoff SPRI beads for >2000bp and resuspend in 150 water. Here we have ssDNA. Considering cfDNA>300bp, the DNA strands have about 6x replicates of each cfDNA sequence. The first 200bp are lost during sequencing, meaning that each cfDNA sequence would be read 5 times. It is also possible to multiplex samples here (by using the BC3 introduced during circularization) to reach the minimum DNA amount of 100 ng required for ONC LIBRARY PREP BY LIGATION (this would allow for skipping an additional PCR step).

The number of samples that can be multiplexed together depends on the amount of DNA obtained after the RCA reaction and on the flow cell sequencing capacity.

Nanopore Library Preparation (e.g., FIG. 1, step 170)

### Option A: ONC Library Prep by Ligation

12. Amplify ssDNA to dsDNA. Prepare following PCR reaction shown in Table 19 below:

TABLE 19
ONC Library Preparation solution.

| | | |
|---|---|---|
| ssDNA solution from Step 11 above. | 15 | μl |
| Epimark HS DNA polymerase buffer 5X | 25 | μl |
| dNTPs 10 mM | 1 | μl |
| Primer Fw on P5 region 10 μM | 1 | μl |
| Primer Rv on P7 region 10 μM | 1 | μl |
| Epimark HS DNA polymerase (it | 0.25 | μl |
| requires 5′−>3′ exo activity) | | |

| | |
|---|---|
| water | Up to 50 μl |

PCR conditions are shown in Table 20 below:

TABLE 20
PCR Conditions.

| | | |
|---|---|---|
| 95° C. | 30 sec | |
| 95° C. | 20 sec | 10x |
| X° C. (45-68° C.) | 30 sec | |
| 68° C. | 3 min | |

### End Repair

13. Start from 100-200 fmol of DNA (if 2000bp minimum of about 125 ng; if 6000bp, a minimum of about 400 ng) on flow cell R9.4 (if flow cell R10.3 use 150-300 fmol).

14. Prepare the following reaction as shown in Table 21 from the NEB NEBNext® Companion Module for Oxford Nanopore Technologies® Ligation Sequencing E7180S:

TABLE 21
Reaction Components.

| Reagent | Volume | |
|---|---|---|
| DNA CS | 1 | μl |
| DNA | 47 | μl |
| NEBNext ® FFPE DNA Repair Buffer | 3.5 | μl |
| NEBNext ® FFPE DNA Repair Mix | 2 | μl |
| Ultra II End-prep reaction buffer | 3.5 | μl |
| Ultra II End-prep enzyme mix | 3 | μl |
| Total | 60 | μl |

15. Incubate 5 min at 20° C., then 5 min at 65° C.

16. Purify DNA with AMPure XP beads and resuspend in 61 μl elution buffer. Store samples O/N at 4° C. if required.

### Adapter Ligation

Depending on the wash buffer (LFB—Long fragment buffer or SFB—short fragment buffer) used, the clean-up step after adapter ligation is designed to either enrich for DNA fragments of >3 kb or purify all fragments equally.

17. Mix in a tube the following as shown in Table 22:

TABLE 22
Reagent mixture.

| Reagent | Volume |
|---|---|
| DNA sample from the previous step | 60 μl |
| Ligation Buffer (LNB) | 25 μl |
| NEBNext ® Quick T4 DNA Ligase | 10 μl |
| Adapter Mix (AMX) | 5 μl |

18. Incubate at RT (Room Temperature) for 10 min.

19. Purify DNA with AMPure XP beads and resuspend in 15 μl elution buffer.

20. Quantify DNA and load on flow cell.

21. Recommended amount of DNA to be loaded for sequencing is 5-50 fmol (for sequencing 2000bp fragments, about 50 ng; for sequencing 6000bp fragments, about 150 ng) of this final prepared library onto R9.4.1 flow cells or 25-75 fmol onto R10.3 flow cells. Loading more than 50 fmol of DNA can have a detrimental effect on throughput.

### Option B: ONC Library Prep by PCR

12. Convert ssDNA into dsDNA by performing one-cycle PCR by preparing the following reaction in Table 23:

TABLE 23
ONC Library preparation by PCR.

| | | |
|---|---|---|
| ssDNA solution from 11. | 15 | μl |
| Epimark HS DNA polymerase buffer 5X | 25 | μl |
| dNTPs 10 mM | 1 | μl |
| Primer Rv on P7 region 10 μM | 1 | μl |
| Epimark HS DNA polymerase (it | 0.25 | μl |
| requires 5′−>3′ exo activity) | | |

| | |
|---|---|
| water | Up to 50 μl |

Reaction conditions in Table 24:

TABLE 24
Reaction Conditions.

| | | |
|---|---|---|
| X° C. (45-68° C.) | 1 min | 1x |
| 68° C. | 3 min | |

### End Repair and Preparation

13. Prepare the following reaction in Table 25 using DNA from Step 11:

TABLE 25
Reaction mixture.

| Reagent | Volume |
|---|---|
| 100 ng fragmented DNA | 50 μl |
| Ultra II End-prep reaction buffer | 7 μl |
| Ultra II End-prep enzyme mix | 3 μl |
| Total | 60 μl |

14. Incubate 5min at 20° C., then 5 min at 65° C.

15. Purify DNA with AMPure beads and resuspend in 160 elution buffer. Quantify DNA concentration.

### PCR Adapter Ligation and Amplification

16. Prepare the following reaction as shown in Table 26:

TABLE 26
PCR mixture.

| Reagent | Volume |
|---|---|
| End-prepped DNA | 15 μl |
| PCR Adapters (PCA) | 10 μl |
| Blunt/TA Ligase Master Mix | 25 μl |
| Total | 50 μl |

17. Incubate 10 min at RT.

18. Purify with AMPure beads and resuspend in 210. Quantify DNA.

19. Prepare the following reaction shown in Table 27:

TABLE 27
Reaction mixture.

| | | | Final concentration |
|---|---|---|---|
| Reagent | Volume | | in 50 μl |
| Adapter ligated DNA, diluted | X | μl | 0.2 ng/μl |
| Nuclease-free water | 24-x | μl | |
| Whole Genome Primers (WGP, at 10 | 1 | μl | |
| μM) | | | |
| LongAmp Hot Start Taq 2x Master Mix | 25 | μl | |
| Total | 50 | μl | |

20. Run PCR under following conditions shown in Table 28:

TABLE 28
PCR reaction conditions.

| Cycle Step | Temperature | | Time | | No. of cycles |
|---|---|---|---|---|---|
| Initial denaturation | 95° | C. | 3 | mins | 1 |
| Denaturation | 95° | C. | 15 | sec | 14 (b) |
| Annealing | 56° | C. (a) | 15 | secs (a) | 14 (b) |
| Extension | 65° | C. (c) | 50 | secs/kb | 14 (b) |
| Final Extension | 65° | C. | 6 | mins | 1 |

| | | | |
|---|---|---|---|
| Hold | 4° | C. | ∞ |

21. Purify with AMPure beads and resuspend in 10 μl in 10 mM Tris-HCl pH 8.0 with 50 mM NaCl.

### Rapid Adapter Ligation

22. Add 1 μl of rapid adaptor (RAP).

23. Incubate 5 min at RT.

Library is ready for loading on Nanopore flow cell for subsequent sequencing (e.g., FIG. 1, step 175) and evaluation of the methylation and/or mutation targets (e.g., FIG. 1, step 180).

### Computer System and Network Architecture

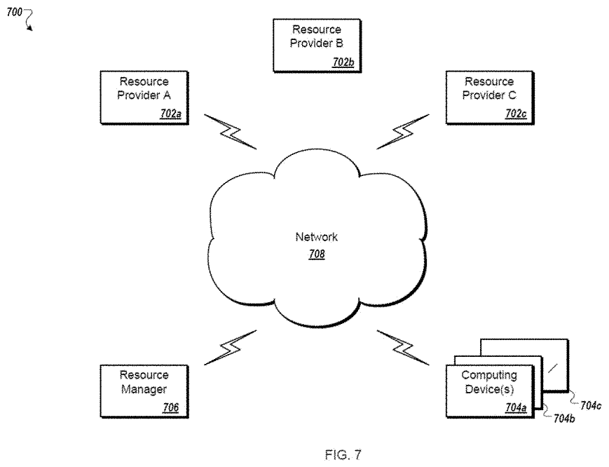

As shown in FIG. 7, an implementation of a network environment 700 for use in providing systems, methods, and architectures described herein is shown and described. In brief overview, referring now to FIG. 7, a block diagram of an exemplary cloud computing environment 700 is shown and described. The cloud computing environment 700 may include one or more resource providers 702a, 702b, 702c (collectively, 702). Each resource provider 702 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 702 may be connected to any other resource provider 702 in the cloud computing environment 700. In some implementations, the resource providers 702 may be connected over a computer network 708. Each resource provider 702 may be connected to one or more computing device 704a, 704b, 704c (collectively, 704), over the computer network 708.

The cloud computing environment 700 may include a resource manager 706. The resource manager 706 may be connected to the resource providers 702 and the computing devices 704 over the computer network 708. In some implementations, the resource manager 706 may facilitate the provision of computing resources by one or more resource providers 702 to one or more computing devices 704. The resource manager 706 may receive a request for a computing resource from a particular computing device 704. The resource manager 706 may identify one or more resource providers 702 capable of providing the computing resource requested by the computing device 704. The resource manager 706 may select a resource provider 702 to provide the computing resource. The resource manager 706 may facilitate a connection between the resource provider 702 and a particular computing device 704. In some implementations, the resource manager 706 may establish a connection between a particular resource provider 702 and a particular computing device 704. In some implementations, the resource manager 706 may redirect a particular computing device 704 to a particular resource provider 702 with the requested computing resource.

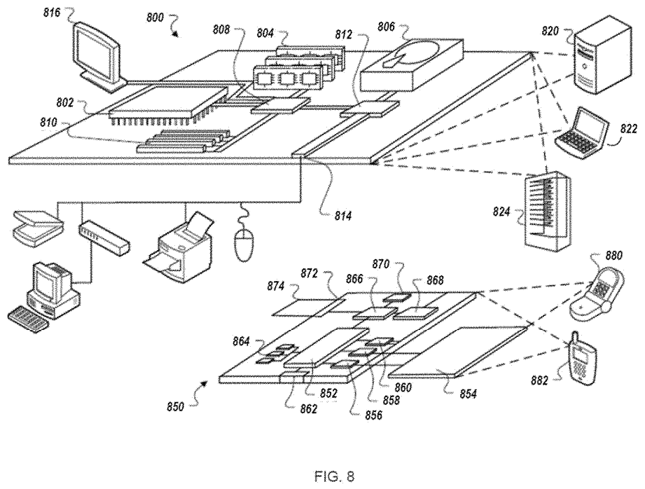

FIG. 8 shows an example of a computing device 800 and a mobile computing device 850 that can be used to implement the techniques described in this disclosure. The computing device 800 is intended to represent various forms of digital computers, such as laptops, desktops, workstations, personal digital assistants, servers, blade servers, mainframes, and other appropriate computers. The mobile computing device 850 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 802), perform one or more methods, such as those described above. The instructions can also be stored by one or more storage devices such as computer- or machine-readable mediums (for example, the memory 804, the storage device 806, or memory on the processor 802).

The high-speed interface 808 manages bandwidth-intensive operations for the computing device 800, while the low-speed interface 812 manages lower bandwidth-intensive operations. Such allocation of functions is an example only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 can execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 858 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may provide communication with the processor 852, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 can be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provide as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. that the instructions, when executed by one or more processing devices (for example, processor 852), perform one or more methods, such as those described above. The instructions can also be stored by one or more storage devices, such as one or more computer- or machine-readable mediums (for example, the memory 864, the expansion memory 874, or memory on the processor 852). In some implementations, the instructions can be received in a propagated signal, for example, over the transceiver 868 or the external interface 862.

The mobile computing device 850 may communicate wireles sly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth®, Wi-Fi™, or other such transceiver (not shown). In addition, a GPS (Global Positioning System) receiver module 870 may provide additional navigation- and location-related wireless data to the mobile computing device 850, which may be used as appropriate by applications running on the mobile computing device 850.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the various modules described herein can be separated, combined or incorporated into single or combined modules. The modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

The various described embodiments of the invention may be used in conjunction with one or more other embodiments unless technically incompatible. It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

### Other Embodiments

While we have described a number of embodiments, it is apparent that our basic disclosure and examples may provide other embodiments that utilize or are encompassed by the compositions and methods described herein. Therefore, it will be appreciated that the scope of is to be defined by that which may be understood from the disclosure and the appended claims rather than by the specific embodiments that have been represented by way of example.

All references cited herein are hereby incorporated by reference.

1. A method comprising:
capturing a subset of deoxyribonucleic acid (DNA) fragments of cell free DNA (cfDNA) with one or more capture probes;
converting said captured DNA fragments into circular DNA; and
amplifying the circular DNA.

2. The method of claim 1, further comprising extracting cfDNA from a biological sample and converting the cfDNA prior to capturing the subset of DNA fragments with the one or more capture probes.

3. The method of claim 2, wherein converting the cfDNA comprises enzymatic treatment of the cfDNA.

4. The method of claim 1 wherein the method comprises adding control DNA molecules to a sample comprising the DNA fragments of cfDNA, wherein the sequence, number of methylated bases, and number of unmethylated bases of the control DNA molecules had been determined prior to addition of the control DNA to the sample.

5. The method of claim 2, wherein the biological sample comprises a member selected from the group consisting of plasma, blood, serum, urine, stool, and tissue.

6. The method of claim 1, wherein the one or more capture probes comprises one or more methylation capture probes and/or one or more mutation capture probes.

7. The method of claim 1, wherein at least one of the one or more capture probes targets a differentially methylated region (DMR) in a genome of interest.

8. The method of claim 1, comprising converting the captured DNA fragments into circular double stranded DNA (dsDNA) and/or circular single stranded DNA (ssDNA) by performing DNA circularization.

9. The method of claim 8, wherein the method comprises converting the captured DNA fragments into circular ssDNA and a portion of the circular ssDNA is complementary to the original cfDNA strand.

10. The method of claim 1, comprising amplifying the circular DNA by performing rolling circle amplification (RCA).

11. The method of claim 1, further comprising sequencing the cfDNA using the amplified circular DNA to produce sequencing results.

12. The method of claim 11, wherein the sequencing step is performed using a third generation sequencing system.

13. The method of claim 11, wherein the method comprises performing sequencing using nanopore sequencing or single molecule real time sequencing (SMRT).

14. The method of claim 11, wherein sequencing the cfDNA comprises producing reads each having length of at least 900 bases.

15. The method of claim 11, further comprising performing (i) methylation target evaluation, or (ii) mutation target evaluation, or (iii) simultaneous methylation target and mutation target evaluation from the sequencing results.

16. The method of claim 11, comprising determining that a subject has a disease or condition.

17. The method of claim 15, wherein the method comprises determining that a subject has a disease or condition based at least in part on the methylation target and/or mutation target evaluation.

18. The method of claim 1, wherein the one or more capture probes are selected and/or are used in a predetermined ratio to enrich for only methylated reads or for only unmethylated reads in one or more specific target regions, thereby reducing (or eliminating) non-informative reads and enhancing a disease-distinguishing signal against background noise.

19. A method comprising:
extracting DNA from a biological sample of a human subject to obtain a DNA sample;
adding control DNA molecules to the DNA sample;
converting unmethylated cytosines to uracils of the DNA in the DNA sample using enzymatic conversion;
adding an index primer to the converted DNA;
amplifying the indexed DNA;
capturing a subset of indexed DNA with one or more capture probes, wherein each of said capture probes are targeted to a pre-determined mutation locus or a pre-determined methylation locus;
converting said captured DNA fragments into circular, single stranded DNA, wherein converting said captured DNA fragments into circular ssDNA comprises binding a splint DNA segment to the indexed DNA;
amplifying the circular, ssDNA using rolling circle amplification;
creating a library of DNA from the amplified, circular ssDNA; and
sequencing the library using third generation sequencing to produce sequencing results.

20. The method of claim 19, wherein sequencing the library comprises producing reads each having length of at least 900 bases.

21. The method of claim 19, wherein the method comprises determining whether a subject has a disease or condition based on the sequencing results.

22. The method of claim 19, further comprising determining the number of methylated cytosines of the control DNA molecules that were converted into uracils.
